SEQUENCE LISTING (179 sequences)

SEQ ID NO: 1 - DNA, 60 bp, Artificial Sequence
  Description of Artificial Sequenceprobe
ttcccccaacctgaccacctcattcttatctttcttctgtttcttcctccccgctgtcat

SEQ ID NO: 2 - DNA, 60 bp, Artificial Sequence
  Description of Artificial Sequenceprobe
gaccatctctccaactcaactcaacctggacactctcttctctgctgagtttgccttgtt

SEQ ID NO: 3 - DNA, 53 bp, Artificial Sequence
  Description of Artificial Sequenceprobe
gaggcatgtctggttcggcgagagcatgaccgatggattccagttcgagtatg

SEQ ID NO: 4 - DNA, 56 bp, Artificial Sequence
  Description of Artificial Sequenceprobe
catgacaactgccgggagggccacgcaggtcgtggtcacctgccagcgactgtctc

SEQ ID NO: 5 - DNA, 60 bp, Artificial Sequence
  Description of Artificial Sequenceprobe
caggggcttctagctgactcgcacagggattctcacaatagccgatatcagaatttgtgt

SEQ ID NO: 6 - DNA, 52 bp, Artificial Sequence
  Description of Artificial Sequenceprobe
cagatgccaggagccctgctgcccacatgcaaggaccagcatctcctgagag

SEQ ID NO: 7 - DNA, 56 bp, Artificial Sequence
  Description of Artificial Sequenceprobe
tattgccgaaccggccgttgtgctacccgtgagtccctctccggggtgtgtgaaat

SEQ ID NO: 8 - DNA, 60 bp, Artificial Sequence
  Description of Artificial Sequenceprobe
atctgtatgacaacccgggatcgtttgcaagtaactgaatccattgcgacattgtgaagg

SEQ ID NO: 9 - DNA, 60 bp, Artificial Sequence
  Description of Artificial Sequenceprobe
acaggatcgtatcatatactcgagacttaccgcatattacagtggatcgattagtgtcaa

SEQ ID NO: 10 - DNA, 54 bp, Artificial Sequence
  Description of Artificial Sequenceprobe
cagcattcctgcactaacggcaactctacgatgtgtccgtgaccctcagagatc

SEQ ID NO: 11 - DNA, 60 bp, Artificial Sequence
  Description of Artificial Sequenceprobe
acaaacccgaaaagagtgtgagctaactagtttccaaagcggagacttccgacttcctta

SEQ ID NO: 12 - DNA, 60 bp, Artificial Sequence
  Description of Artificial Sequenceprobe
acttgcacgtgtccctgaattccgctgactctaatttatgaggatgccgaactctgatgg

SEQ ID NO: 13 - DNA, 53 bp, Artificial Sequence
  Description of Artificial Sequenceprobe
ggaactgacgcagctacgccatgaactggagcggcagaacaatgaataccaag

SEQ ID NO: 14 - DNA, 53 bp, Artificial Sequence
  Description of Artificial Sequenceprobe
ccgcggccatggcgtacgcctatctcttcaagtacatcataatcggcgacaca

SEQ ID NO: 15 - DNA, 60 bp, Artificial Sequence
  Description of Artificial Sequenceprobe
tctgttgccgaaatagctggtcctttttcgggagttagatgtatagagtgtttgtatgta

SEQ ID NO: 16 - DNA, 54 bp, Artificial Sequence
  Description of Artificial Sequenceprobe
caggaccggcccgaccgagacaatgacctacggttggcccagcacatcacctat

SEQ ID NO: 17 - DNA, 52 bp, Artificial Sequence
  Description of Artificial Sequenceprobe
gtgcgctgtgctaggtcagcaccacaactaccagaactggagggtgtacgac

SEQ ID NO: 18 - DNA, 52 bp, Artificial Sequence
  Description of Artificial Sequenceprobe
gaagaagactcgagctgccagggaaggcgaccgacgagataatcgccttcgg

SEQ ID NO: 19 - DNA, 52 bp, Artificial Sequence
  Description of Artificial Sequenceprobe
cactggccgaggataagcccgtccctgtcccacattctagccccactatgcg

SEQ ID NO: 20 - DNA, 60 bp, Artificial Sequence
  Description of Artificial Sequenceprobe
actgcagcgtatcaaactaaaaggcaccattgacgtgtcaaagctggttacggggactgt

SEQ ID NO: 21 - DNA, 60 bp, Artificial Sequence
  Description of Artificial Sequenceprobe
taataccactacccgtgaattatatggcctgacaatatgaattaggtgtactgtactgaa

SEQ ID NO: 22 - DNA, 60 bp, Artificial Sequence
  Description of Artificial Sequenceprobe
tagtcttccccagtcctcattcccagctgcctcttcctactgcttccgtctatcaaaaag

SEQ ID NO: 23 - DNA, 54 bp, Artificial Sequence
  Description of Artificial Sequenceprobe
tggccacatgggaccaagcggaaccagaacaaggtcccgacaggggtagacgtt

SEQ ID NO: 24 - DNA, 60 bp, Artificial Sequence
  Description of Artificial Sequenceprobe
agtcaaagtgcgagtcaacacccatggcattttcaccatctctacggcatctatggtgga

SEQ ID NO: 25 - DNA, 60 bp, Artificial Sequence
  Description of Artificial Sequenceprobe
gtcttagaacctcaactgacatatagcattgggcacactccagcagacgcccgaattcaa

SEQ ID NO: 26 - DNA, 52 bp, Artificial Sequence
  Description of Artificial Sequenceprobe
tgggcccaaggctcatgcacacgctacctattgtggcacggagagtaaggac

SEQ ID NO: 27 - DNA, 60 bp, Artificial Sequence
  Description of Artificial Sequenceprobe
ccaccacgcttggccgggatagtatatttttatagcacttcccctactgattgctgcctt

SEQ ID NO: 28 - DNA, 60 bp, Artificial Sequence
  Description of Artificial Sequenceprobe
gaaatattgcggtaccaaggttggctgtaccaacatcgcctatccaaccttagtggtgga

SEQ ID NO: 29 - DNA, 60 bp, Artificial Sequence
  Description of Artificial Sequenceprobe
atgacgattactactattatggtccacctcatatgccccctccaacaagaggtcgagggc

SEQ ID NO: 30 - DNA, 60 bp, Artificial Sequence
  Description of Artificial Sequenceprobe
tatgtctgtgtatgtataggggaaagaagggatccctaactgttcccttatctgttttct

SEQ ID NO: 31 - DNA, 60 bp, Artificial Sequence
  Description of Artificial Sequenceprobe
ctctgcaagctatcgggatcgtagtgaaaccgatagagctggttatgctaatggcagtgg

SEQ ID NO: 32 - DNA, 52 bp, Artificial Sequence
  Description of Artificial Sequenceprobe
gccggcggcatcgagactatcgctaatgagtatagcgaccgctgcacgccgg

SEQ ID NO: 33 - DNA, 52 bp, Artificial Sequence
  Description of Artificial Sequenceprobe
aaaggcctatcggagctatgtgctgcaccgctccatggcactctctaacacc

SEQ ID NO: 34 - DNA, 56 bp, Artificial Sequence
  Description of Artificial Sequenceprobe
aagaacacgctgtatctgcaaatgcacaggctgagagccgaggacacggccgtata

SEQ ID NO: 35 - DNA, 60 bp, Artificial Sequence
  Description of Artificial Sequenceprobe
gcccggcgatattgtcattattgatcagttcattgacaggactatttgccacgacatttc

SEQ ID NO: 36 - DNA, 53 bp, Artificial Sequence
  Description of Artificial Sequenceprobe
ccctcacatcgcccctcggctacaacgcttctctctcgaggcatcgtagctcg

SEQ ID NO: 37 - DNA, 56 bp, Artificial Sequence
  Description of Artificial Sequenceprobe
ggaggactacgacagcttccttatgtacagcgatgacgttctacgctctccatcgg

SEQ ID NO: 38 - DNA, 60 bp, Artificial Sequence
  Description of Artificial Sequenceprobe
ctgcctgacctttaaaaggccatcgatattctttgctccttgccataggacttgtattgt

SEQ ID NO: 39 - DNA, 60 bp, Artificial Sequence
  Description of Artificial Sequenceprobe
cctttgttaacactccttaccaagtccacacgactgacgatgacacggaatgcagtctgg

SEQ ID NO: 40 - DNA, 60 bp, Artificial Sequence
  Description of Artificial Sequenceprobe
cagcaccgccgaagggctctccttgtcgctcataaagtccgagtgcggcgatcttcaaga

SEQ ID NO: 41 - DNA, 60 bp, Artificial Sequence
  Description of Artificial Sequenceprobe
ggaagtttacgcctgatagccaaacagggaaaaagttaatggcgaagtgtcgaatgctta

SEQ ID NO: 42 - DNA, 60 bp, Artificial Sequence
  Description of Artificial Sequenceprobe
aaaggccggatgctaggtgatgtgctaatggatcagaacgtattgcctggagtagggaac

SEQ ID NO: 43 - DNA, 56 bp, Artificial Sequence
  Description of Artificial Sequenceprobe
agcccagttgtagtaggtgccagtcagtcaaggcaggggccctctctccgtcaata

SEQ ID NO: 44 - DNA, 54 bp, Artificial Sequence
  Description of Artificial Sequenceprobe
catgttggccaagctagtctcgaactactgacttcgggtgatctgccctcctcg

SEQ ID NO: 45 - DNA, 53 bp, Artificial Sequence
  Description of Artificial Sequenceprobe
tcgcccgtcacgcaccgcacgttcgtggggaacctggcgctaaaccattcgta

SEQ ID NO: 46 - DNA, 54 bp, Artificial Sequence
  Description of Artificial Sequenceprobe
ctcaggatacgaagacatttgacgttgcgctgtccgaggagctccatgcggcac

SEQ ID NO: 47 - DNA, 60 bp, Artificial Sequence
  Description of Artificial Sequenceprobe
gctgatccaccgatacgtggagctcttagacaaatactttggcagtgtgtgcgagctgga

SEQ ID NO: 48 - DNA, 60 bp, Artificial Sequence
  Description of Artificial Sequenceprobe
ctctcaaaccaactcgtactgtcgaggtggattctaaagcagcagaaattggtaagaaag

SEQ ID NO: 49 - DNA, 60 bp, Artificial Sequence
  Description of Artificial Sequenceprobe
ctcagaaacggacgacgcaagaagtgcaagcgacttctagaattcagaaccgaaagtgga

SEQ ID NO: 50 - DNA, 60 bp, Artificial Sequence
  Description of Artificial Sequenceprobe
aactggtcctgcaattactatgaagtcaaaaattaaactagactatgtctccaactcagt

SEQ ID NO: 51 - DNA, 21 bp, Artificial Sequence
  Description of Artificial Sequenceprimer
gagaagcacagcatttctgag

SEQ ID NO: 52 - DNA, 21 bp, Artificial Sequence
  Description of Artificial Sequenceprimer
tgctctttaaagccttaggcc

SEQ ID NO: 53 - DNA, 20 bp, Artificial Sequence
  Description of Artificial Sequenceprimer
aatcctggctactgtgtgag

SEQ ID NO: 54 - DNA, 19 bp, Artificial Sequence
  Description of Artificial Sequenceprimer
tccaaagactggggtaggt

SEQ ID NO: 55 - DNA, 20 bp, Artificial Sequence
  Description of Artificial Sequenceprimer
ctactaccgggctgatgatg

SEQ ID NO: 56 - DNA, 20 bp, Artificial Sequence
  Description of Artificial Sequenceprimer
ggaggacttggtggttttgt

SEQ ID NO: 57 - DNA, 21 bp, Artificial Sequence
  Description of Artificial Sequenceprimer
cacgaatatcaggctagagac

SEQ ID NO: 58 - DNA, 22 bp, Artificial Sequence
  Description of Artificial Sequenceprimer
cacatttacaatggctttggag

SEQ ID NO: 59 - DNA, 18 bp, Artificial Sequence
  Description of Artificial Sequenceprimer
ggctccagtgaaacttgg

SEQ ID NO: 60 - DNA, 19 bp, Artificial Sequence
  Description of Artificial Sequenceprimer
caaggtaacgtgagcactt

SEQ ID NO: 61 - DNA, 20 bp, Artificial Sequence
  Description of Artificial Sequenceprimer
acccattacggctactcctc

SEQ ID NO: 62 - DNA, 21 bp, Artificial Sequence
  Description of Artificial Sequenceprimer
aaggggtgttgcttttattgc

SEQ ID NO: 63 - DNA, 17 bp, Artificial Sequence
  Description of Artificial Sequenceprimer
cagccatgaggaccatc

SEQ ID NO: 64 - DNA, 21 bp, Artificial Sequence
  Description of Artificial Sequenceprimer
tagaaagacacaaggtacaca

SEQ ID NO: 65 - DNA, 20 bp, Artificial Sequence
  Description of Artificial Sequenceprimer
gagaaaggaaccgagcttgt

SEQ ID NO: 66 - DNA, 20 bp, Artificial Sequence
  Description of Artificial Sequenceprimer
tggtagtaagggcaagctga

SEQ ID NO: 67 - DNA, 20 bp, Artificial Sequence
  Description of Artificial Sequenceprimer
tggcctacctataactggaa

SEQ ID NO: 68 - DNA, 20 bp, Artificial Sequence
  Description of Artificial Sequenceprimer
aatggatgttctgcctgaag

SEQ ID NO: 69 - DNA, 20 bp, Artificial Sequence
  Description of Artificial Sequenceprimer
gaaggccacctgaacctaac

SEQ ID NO: 70 - DNA, 20 bp, Artificial Sequence
  Description of Artificial Sequenceprimer
ccatctgaagactccgacag

SEQ ID NO: 71 - DNA, 21 bp, Artificial Sequence
  Description of Artificial Sequenceprimer
gatcttcaagtcctcatcacc

SEQ ID NO: 72 - DNA, 20 bp, Artificial Sequence
  Description of Artificial Sequenceprimer
accagctttaactcaccttc

SEQ ID NO: 73 - DNA, 21 bp, Artificial Sequence
  Description of Artificial Sequenceprimer
gtacgtccagaaaaggctatg

SEQ ID NO: 74 - DNA, 19 bp, Artificial Sequence
  Description of Artificial Sequenceprimer
ggacttagggtcatcggaa

SEQ ID NO: 75 - DNA, 18 bp, Artificial Sequence
  Description of Artificial Sequenceprimer
aggtgacatccacgaact

SEQ ID NO: 76 - DNA, 22 bp, Artificial Sequence
  Description of Artificial Sequenceprimer
gtaggaaagtagagctttaccc

SEQ ID NO: 77 - DNA, 19 bp, Artificial Sequence
  Description of Artificial Sequenceprimer
cagttcgtccggcttcctc

SEQ ID NO: 78 - DNA, 24 bp, Artificial Sequence
  Description of Artificial Sequenceprimer
atgaagatgagtccatgttctcgt

SEQ ID NO: 79 - DNA, 17 bp, Artificial Sequence
  Description of Artificial Sequenceprimer
actgcacccatgagact

SEQ ID NO: 80 - DNA, 20 bp, Artificial Sequence
  Description of Artificial Sequenceprimer
gttcatggtgatgcctacaa

SEQ ID NO: 81 - DNA, 20 bp, Artificial Sequence
  Description of Artificial Sequenceprimer
gtggagaactgaccttagag

SEQ ID NO: 82 - DNA, 21 bp, Artificial Sequence
  Description of Artificial Sequenceprimer
tcctttgacatctccattagc

SEQ ID NO: 83 - DNA, 19 bp, Artificial Sequence
  Description of Artificial Sequenceprimer
ctcctcctgttcctatgtg

SEQ ID NO: 84 - DNA, 18 bp, Artificial Sequence
  Description of Artificial Sequenceprimer
gcaggagaagcatcgatg

SEQ ID NO: 85 - DNA, 20 bp, Artificial Sequence
  Description of Artificial Sequenceprimer
agagtgcgagaacaacgaat

SEQ ID NO: 86 - DNA, 21 bp, Artificial Sequence
  Description of Artificial Sequenceprimer
actaaaggtcaggaaagggaa

SEQ ID NO: 87 - DNA, 20 bp, Artificial Sequence
  Description of Artificial Sequenceprimer
cttcacctgcagagtccttt

SEQ ID NO: 88 - DNA, 20 bp, Artificial Sequence
  Description of Artificial Sequenceprimer
gatagagggtcatgcagtgg

SEQ ID NO: 89 - DNA, 20 bp, Artificial Sequence
  Description of Artificial Sequenceprimer
caactcctcacaacccttcc

SEQ ID NO: 90 - DNA, 20 bp, Artificial Sequence
  Description of Artificial Sequenceprimer
ttcttggtgaggtatttggc

SEQ ID NO: 91 - DNA, 19 bp, Artificial Sequence
  Description of Artificial Sequenceprimer
gaagaaacgcctcaaaagc

SEQ ID NO: 92 - DNA, 24 bp, Artificial Sequence
  Description of Artificial Sequenceprimer
ttttctttaatacagttgccatct

SEQ ID NO: 93 - DNA, 20 bp, Artificial Sequence
  Description of Artificial Sequenceprimer
caagctgtcactccccatac

SEQ ID NO: 94 - DNA, 20 bp, Artificial Sequence
  Description of Artificial Sequenceprimer
gggaggtcaggctttagaga

SEQ ID NO: 95 - DNA, 19 bp, Artificial Sequence
  Description of Artificial Sequenceprimer
tggtttctgccatccggaa

SEQ ID NO: 96 - DNA, 22 bp, Artificial Sequence
  Description of Artificial Sequenceprimer
cttgatcctctctagcgcgtaa

SEQ ID NO: 97 - DNA, 18 bp, Artificial Sequence
  Description of Artificial Sequenceprimer
caatactttccccggcat

SEQ ID NO: 98 - DNA, 18 bp, Artificial Sequence
  Description of Artificial Sequenceprimer
cctaactgccagaccaag

SEQ ID NO: 99 - DNA, 20 bp, Artificial Sequence
  Description of Artificial Sequenceprimer
cgcagaccttgtgatattcc

SEQ ID NO: 100 - DNA, 23 bp, Artificial Sequence
  Description of Artificial Sequenceprimer
cgattccctctcatttattcctt

SEQ ID NO: 101 - DNA, 20 bp, Artificial Sequence
  Description of Artificial Sequenceprimer
gaggatgatgagctgctaca

SEQ ID NO: 102 - DNA, 22 bp, Artificial Sequence
  Description of Artificial Sequenceprimer
ctacacacttttattggagggg

SEQ ID NO: 103 - DNA, 23 bp, Artificial Sequence
  Description of Artificial Sequenceprimer
aactcatttacggataggacttt

SEQ ID NO: 104 - DNA, 20 bp, Artificial Sequence
  Description of Artificial Sequenceprimer
ttcctgtcctattggtacca

SEQ ID NO: 105 - DNA, 20 bp, Artificial Sequence
  Description of Artificial Sequenceprimer
aaatgctacactccaagggc

SEQ ID NO: 106 - DNA, 21 bp, Artificial Sequence
  Description of Artificial Sequenceprimer
cagaaaatagcaagcaggaag

SEQ ID NO: 107 - DNA, 22 bp, Artificial Sequence
  Description of Artificial Sequenceprimer
aatgggctgatcctatagaaga

SEQ ID NO: 108 - DNA, 20 bp, Artificial Sequence
  Description of Artificial Sequenceprimer
ctctttgttgttgggcacct

SEQ ID NO: 109 - DNA, 21 bp, Artificial Sequence
  Description of Artificial Sequenceprimer
acctcatctccaaactgctca

SEQ ID NO: 110 - DNA, 25 bp, Artificial Sequence
  Description of Artificial Sequenceprimer
aaaaagcttcagcctttattaaaca

SEQ ID NO: 111 - DNA, 21 bp, Artificial Sequence
  Description of Artificial Sequenceprimer
ctcagaggattatgtgcaccg

SEQ ID NO: 112 - DNA, 20 bp, Artificial Sequence
  Description of Artificial Sequenceprimer
aggaggaggagggtattggt

SEQ ID NO: 113 - DNA, 22 bp, Artificial Sequence
  Description of Artificial Sequenceprimer
cagtacccactggaaggactta

SEQ ID NO: 114 - DNA, 22 bp, Artificial Sequence
  Description of Artificial Sequenceprimer
tctccttcagtttggacaaaag

SEQ ID NO: 115 - DNA, 20 bp, Artificial Sequence
  Description of Artificial Sequenceprimer
agaacttcaatgtggccacg

SEQ ID NO: 116 - DNA, 20 bp, Artificial Sequence
  Description of Artificial Sequenceprimer
catactggtggcttcatctt

SEQ ID NO: 117 - DNA, 21 bp, Artificial Sequence
  Description of Artificial Sequenceprimer
gtgggtctcagctattagtgg

SEQ ID NO: 118 - DNA, 23 bp, Artificial Sequence
  Description of Artificial Sequenceprimer
ggatttcgcacagtaatatacgg

SEQ ID NO: 119 - DNA, 20 bp, Artificial Sequence
  Description of Artificial Sequenceprimer
ctcgcttggttcccttagtc

SEQ ID NO: 120 - DNA, 20 bp, Artificial Sequence
  Description of Artificial Sequenceprimer
atccctgcaggaaaaatcat

SEQ ID NO: 121 - DNA, 20 bp, Artificial Sequence
  Description of Artificial Sequenceprimer
atgtcaagggtgcatcagtc

SEQ ID NO: 122 - DNA, 20 bp, Artificial Sequence
  Description of Artificial Sequenceprimer
gaacccttctgaacctggtc

SEQ ID NO: 123 - DNA, 20 bp, Artificial Sequence
  Description of Artificial Sequenceprimer
tctggccttcaatgtcgtct

SEQ ID NO: 124 - DNA, 20 bp, Artificial Sequence
  Description of Artificial Sequenceprimer
tgtggtcgagtgtgagtgtt

SEQ ID NO: 125 - DNA, 20 bp, Artificial Sequence
  Description of Artificial Sequenceprimer
atgtccatgtgaacgctact

SEQ ID NO: 126 - DNA, 20 bp, Artificial Sequence
  Description of Artificial Sequenceprimer
ccaagcctctggttctgatg

SEQ ID NO: 127 - DNA, 20 bp, Artificial Sequence
  Description of Artificial Sequenceprimer
caagagtggtggatcggttc

SEQ ID NO: 128 - DNA, 20 bp, Artificial Sequence
  Description of Artificial Sequenceprimer
tcaacaagtggagaaggcaa

SEQ ID NO: 129 - DNA, 20 bp, Artificial Sequence
  Description of Artificial Sequenceprimer
tgatggcctatccatttcag

SEQ ID NO: 130 - DNA, 20 bp, Artificial Sequence
  Description of Artificial Sequenceprimer
aacatctttgcctgcgataa

SEQ ID NO: 131 - DNA, 20 bp, Artificial Sequence
  Description of Artificial Sequenceprimer
aagcaacaacagcaggagtc

SEQ ID NO: 132 - DNA, 20 bp, Artificial Sequence
  Description of Artificial Sequenceprimer
tgtgaaatccagacccagac

SEQ ID NO: 133 - DNA, 20 bp, Artificial Sequence
  Description of Artificial Sequenceprimer
atttcggaatgaaaggcttc

SEQ ID NO: 134 - DNA, 20 bp, Artificial Sequence
  Description of Artificial Sequenceprimer
ccctgctagatgtccaactg

SEQ ID NO: 135 - DNA, 20 bp, Artificial Sequence
  Description of Artificial Sequenceprimer
gcttataccctgttcccaaa

SEQ ID NO: 136 - DNA, 23 bp, Artificial Sequence
  Description of Artificial Sequenceprimer
acacagctccatcataatttcat

SEQ ID NO: 137 - DNA, 24 bp, Artificial Sequence
  Description of Artificial Sequenceprimer
gctcagtgctagtctgttgtgtag

SEQ ID NO: 138 - DNA, 19 bp, Artificial Sequence
  Description of Artificial Sequenceprimer
ccctggcctcaagtgatcc

SEQ ID NO: 139 - DNA, 19 bp, Artificial Sequence
  Description of Artificial Sequenceprimer
gaattcaccaagcgttgga

SEQ ID NO: 140 - DNA, 22 bp, Artificial Sequence
  Description of Artificial Sequenceprimer
gctgactttcaatagatcgcag

SEQ ID NO: 141 - DNA, 20 bp, Artificial Sequence
  Description of Artificial Sequenceprimer
tgttcctctggaactggaga

SEQ ID NO: 142 - DNA, 20 bp, Artificial Sequence
  Description of Artificial Sequenceprimer
ccctccttttgctcaagaat

SEQ ID NO: 143 - DNA, 19 bp, Artificial Sequence
  Description of Artificial Sequenceprimer
gaaagcccaagatgtgcag

SEQ ID NO: 144 - DNA, 19 bp, Artificial Sequence
  Description of Artificial Sequenceprimer
gagtcccaccaagaacagg

SEQ ID NO: 145 - DNA, 23 bp, Artificial Sequence
  Description of Artificial Sequenceprimer
agagtaaactttgggccatatga

SEQ ID NO: 146 - DNA, 24 bp, Artificial Sequence
  Description of Artificial Sequenceprimer
accttttttgctatctttctctgt

SEQ ID NO: 147 - DNA, 20 bp, Artificial Sequence
  Description of Artificial Sequenceprimer
cttgaatcatgggtgacgtt

SEQ ID NO: 148 - DNA, 20 bp, Artificial Sequence
  Description of Artificial Sequenceprimer
gtttggtgcgattatgtggt

SEQ ID NO: 149 - DNA, 23 bp, Artificial Sequence
  Description of Artificial Sequenceprimer
ctcaggattcaagaaatggaagg

SEQ ID NO: 150 - DNA, 22 bp, Artificial Sequence
  Description of Artificial Sequenceprimer
gtgaaggtactgaagatcagcg

SEQ ID NO: 151 - DNA, 60 bp, Artificial Sequence
  Description of Artificial Sequenceprobe
ccatagtcaatgatggtttaataggtaaaccaaaccctataaacctgacctcctttatgg

SEQ ID NO: 152 - DNA, 60 bp, Artificial Sequence
  Description of Artificial Sequenceprobe
ccaactgtcaaccttgacaaattgtggactttggtcagtgaacagacacgggtgaatgct

SEQ ID NO: 153 - DNA, 60 bp, Artificial Sequence
  Description of Artificial Sequenceprobe
gagtgtaaggcgtacggtgagaacattgggtacagtgagaaagaccgttttcagggacgt

SEQ ID NO: 154 - DNA, 60 bp, Artificial Sequence
  Description of Artificial Sequenceprobe
ccaccccactcttaatcagtggtggaagaacggtctcagaactgtttgtttcaattggcc

SEQ ID NO: 155 - DNA, 56 bp, Artificial Sequence
  Description of Artificial Sequenceprobe
ctctgatcgtaggaattgaggagtgtcccgccttgtggctgagaactggacagtgg

SEQ ID NO: 156 - DNA, 55 bp, Artificial Sequence
  Description of Artificial Sequenceprobe
tcatccgccgagactatctgcactacatccgcaagtacaaccgcttcgagaagcg

SEQ ID NO: 157 - DNA, 60 bp, Artificial Sequence
  Description of Artificial Sequenceprobe
acatccagcagtggtcattcgacaacgaaagtcataccgtagaaaagatggcgtgtttct

SEQ ID NO: 158 - DNA, 22 bp, Artificial Sequence
  Description of Artificial Sequenceprimer
aattagcagtttagaatggagg

SEQ ID NO: 159 - DNA, 20 bp, Artificial Sequence
  Description of Artificial Sequenceprimer
tgggctgccaacatgccatc

SEQ ID NO: 160 - DNA, 22 bp, Artificial Sequence
  Description of Artificial Sequenceprimer
ggcaagcgagatgaagataagg

SEQ ID NO: 161 - DNA, 21 bp, Artificial Sequence
  Description of Artificial Sequenceprimer
agactatccacctttgggtcg

SEQ ID NO: 162 - DNA, 22 bp, Artificial Sequence
  Description of Artificial Sequenceprimer
ttgagcgcacctaaccactggt

SEQ ID NO: 163 - DNA, 22 bp, Artificial Sequence
  Description of Artificial Sequenceprimer
acattcagactgagcgtgccta

SEQ ID NO: 164 - DNA, 22 bp, Artificial Sequence
  Description of Artificial Sequenceprimer
ttcaagatgtcgaagcgaggac

SEQ ID NO: 165 - DNA, 21 bp, Artificial Sequence
  Description of Artificial Sequenceprimer
ctgtatccaattctgtactgc

SEQ ID NO: 166 - DNA, 20 bp, Artificial Sequence
  Description of Artificial Sequenceprimer
tgtagtagcccgatcgcacc

SEQ ID NO: 167 - DNA, 20 bp, Artificial Sequence
  Description of Artificial Sequenceprimer
aggtcccatacgtatgacag

SEQ ID NO: 168 - DNA, 25 bp, Artificial Sequence
  Description of Artificial Sequenceprimer
gatgcattgttatcattaaccagtc

SEQ ID NO: 169 - DNA, 23 bp, Artificial Sequence
  Description of Artificial Sequenceprimer
gagaggaaacatggtcacaccca

SEQ ID NO: 170 - DNA, 21 bp, Artificial Sequence
  Description of Artificial Sequenceprimer
gatctggacgtccctgaagca

SEQ ID NO: 171 - DNA, 25 bp, Artificial Sequence
  Description of Artificial Sequenceprimer
tgtaatggcagaacctttcatctcg

SEQ ID NO: 172 - DNA, 20 bp, Artificial Sequence
  Description of Artificial Sequenceprimer
aaaattcggccagggttctc

SEQ ID NO: 173 - DNA, 20 bp, Artificial Sequence
  Description of Artificial Sequenceprimer
ggatgccaaatgctgtcaag

SEQ ID NO: 174 - DNA, 22 bp, Artificial Sequence
  Description of Artificial Sequenceprimer
gcctaccaagactttgagaatc

SEQ ID NO: 175 - DNA, 21 bp, Artificial Sequence
  Description of Artificial Sequenceprimer
gatgactgtcaggatgttgct

SEQ ID NO: 176 - DNA, 25 bp, Artificial Sequence
  Description of Artificial Sequenceprimer
gagcatttagtccaacttaatgaaa

SEQ ID NO: 177 - DNA, 20 bp, Artificial Sequence
  Description of Artificial Sequenceprimer
ggactgcttcagtttctctg

SEQ ID NO: 178 - DNA, 22 bp, Artificial Sequence
  Description of Artificial Sequenceprimer
gacttaagcatctgcaatggtg

SEQ ID NO: 179 - DNA, 22 bp, Artificial Sequence
  Description of Artificial Sequenceprimer
cttttccttattggctccagaa

(19) United States
(12) Patent Application Publication
(10) Pub. No.: US 2009/0011413 A1
(43) Pub. Date: Jan. 8, 2009

(54) Method for screening colon cancer cells and gene set used for examination of colon cancer

(75) Inventors: Mie Ishii, Machida-shi (JP); Nobuko Yamamoto, Yokohama-shi (JP); Masahiro Kawaguchi, Atsugi-shi (JP); Tetsuo Okabe, Yokohama-shi (JP); Hiroki Sasaki, Tokyo (JP); Satoshi Yajima, Tokyo (JP); Yasuhiro Matsumura, Tokyo (JP); Hisayuki Matsushita, Kashiwa-shi (JP); Hiroyuki Tsunoda, Kodaira-shi (JP); Kunio Harada, Hachioji-shi (JP)

Correspondence Address: FITZPATRICK CELLA HARPER & SCINTO, 30 ROCKEFELLER PLAZA, NEW YORK, NY, 10112, US

(73) Assignees: CANON KABUSHIKI KAISHA, Tokyo (JP); PRESIDENT OF NATIONAL CANCER CENTER, Tokyo (JP)

(21) Appl. No.: 11/808,943

(22) Filed: Jun. 14, 2007

Related U.S. Application Data
(63) Continuation-in-part of application No. 11/637,087, filed on Dec. 12, 2006, now abandoned.

(30) Foreign Application Priority Data
Dec. 14, 2005 (JP) 2005-360974

(51) Int. Cl. C12Q 1/68; C07H 21/00; C12N 11/00

(52) U.S. Cl. 435/6; 536/24.33; 536/24.31; 435/174

(57) ABSTRACT

Colon cancer cells in a sample are screened by analyzing the amount of expression of at least 2 or more genes or products thereof selected from the group of genes listed in Tables 1 and 30. As compared to conventional method, patients having colon cancer can be detected with higher accuracy. Colon cancer cells in stool are also screened by analyzing expression of genes selected from the group of genes listed in Table 37.

This is a continuation-in-part application of the U.S. patent application Ser. No. 11/637,087 filed on Dec. 12, 2006.

### BACKGROUND OF THE INVENTION

1. Field of the Invention

The present invention relates to an examination method for early colon cancer. Stated more in detail, the present invention relates to a method for screening colon cancer cells in which the expression amount of a specific set of genes in a sample (blood, stool, and the like) is used as an indicator. The present invention also relates to primers, probes, and immobilized samples for this method.

2. Description of the Related Art

The most frequent cause of cancer death in Japanese is stomach cancer. However, in recent years, the number of cases of stomach cancer has been decreasing, and instead, colon cancer has shown a dramatic increase. The ratio of colon cancer among all cancer deaths has been increasing annually from 1955. It is said that in the 21st century, the number of colon cancer deaths will surpass that of stomach cancer deaths to become the top.

On the other hand, colon cancer advances relatively slowly. Even in advanced cancer cases, as long as a complete curative resection is conducted, prognosis is relatively good. Five year survival rates, for example, for Dukes' A, Dukes' B and Dukes' C. is 95%, 80% and 50-60%, respectively. However, there are an ignorable number of cases where no or little subjective symptom appears until a fairly advanced stage and at the time of definitive diagnosis, the cancer has already metastasized or become invasive and resection is no longer possible. Therefore, early detection is strongly needed (see cancer statistics by the National Cancer Center, Tokyo).

Currently, the main method that is used for screening of colon cancer is a fecal occult blood test. In a fecal occult blood test, hemoglobin in blood is chemically measured to detect bleeding from the surface of colonic lumen which cannot be seen by the naked eye can be detected. This method is extremely sensitive, and even a small amount of blood in the stool can be detected. However, while the chemical occult blood test has good sensitivity, this test is not specific to human hemoglobin. False positives are seen when there is a reaction with meat or green vegetables that is eaten or due to medications. Prior to examination, strict dietary restriction is required.

In recent years, an immunological fecal occult blood reaction method has been developed. This method specifically detects human hemoglobin in stool using an antibody and is currently used in actual examination. While the immunologic fecal occult blood reaction specifically detects hemoglobin in stool, hemoglobin easily breaks down in stool, and as a result, there is the problem that, with this immunologic method, hemoglobin that has been broken down cannot be measured.

In addition, the positivity rate for advanced cancers is 90% with this method, but for all stages, which combines early cancers and advanced cancers, the positivity rate is only 50% (Launoy G et al., Int. J. Cancer 1997, 73:220-224). In other words, there is the possibility that one out of two colon cancer cases will be missed. In addition, because this is a detection method which confirms the presence of bleeding, this test is positive for reasons other than cancers, such as hemorrhoids. The probability of having colon cancer among people with positive reaction (positive predictive value) is only approximately 1-2% (Mandel J S et al., N. Engl. J. Med., 2000, 343 (22): 1603-1607). Furthermore, false positive rate (the probability of the test being positive in healthy individuals) is between 5-10%. Further improvement is desired.

On the other hand, diagnosis methods using tumor markers have been proposed. Tumor markers for colon cancer include carcinoembryonic antigen (CEA), CA19-9, NCC-ST-439, STN, and the like. These are used for determining treatment effectiveness and for monitoring of recurrence (Okura, Hisanao et al, Tumor markers for colon cancer, CRC 1(4) 42-47, 1992). There has also been a research into methods which target mutations of DNA (K-RAS, P53, APC, and the like) in stool. However, there are difficulties in implementing these methods targeting mutations in DNA in stool, and these methods are still only in their research stage.

In those methods relying on tumor markers, the tumor marker positivity rates, even with Dukes' C for which curative resection is possible, are only 36%, 30%, 35% and 21% for CEA, CA19-9, NCC-ST-439 and STN, respectively. Thus, it cannot be said that these tumor markers are adequate for early colon cancers (Okura, Hisanao et al., Tumor markers for colon cancer, CRC 1(4), 42-47, 1992).

### SUMMARY OF THE INVENTION

The object of the present invention is to provide an early diagnosis method for colon cancer in which colon cancer patients are detected with high precision as compared to the prior method.

The present inventors have conducted intensive study in order to solve the above problems. The present inventors have then identified genes which are closely associated with colon cancer cells. The present inventors have discovered further that, by measuring expression levels of those genes, colon cancer patients can be detected with high precision.

In addition, the present inventors have discovered a method for screening cancer cells, especially colon cancer cells, by analyzing gene expression of cells collected from stool, as well as a gene set for the gene expression analysis.

As a probe for determining the expression levels of those genes, partial base sequences specific thereto have been identified. In addition, primers which can specifically amplify very small amounts of mRNAs of those genes in a sample have been designed.

In addition, a solid phase carrier of those probes has been provided, and by reacting it with labeled cDNAs in a multiplex RT-PCR (where a plurality of cDNAs are amplified by PCR in one tube), a method for simultaneously measuring the expression levels of a plurality of genes has been developed.

In other words, the present invention provides a method for screening of colon cancer cells in a sample by analyzing an amount of expression of at least 2 or more genes, or products thereof, selected from the group of genes listed in Table 1.

Of the group of genes listed in Table 1, the genes listed in Tables 26 and 28 are genes which particularly differentiate colon cancer from hemorrhoids. Even if blood is contained in a sample, they are suitably used for screening, or judging the presence or absence of, cancer cells.

In the present invention, the expression amount of a gene is analyzed by measuring an amount of a mRNA in a sample. The expression amount of a gene product, on the other hand, is analyzed by using an antibody against the gene product. As a sample, a stool smear or the like obtained from a subject is used. When a stool smear is used, in order to measure the expression levels of respective genes in colon cancer cells released in the stool, a test sample is prepared in which a buffer is added at room temperature to the naturally excreted stool, and impurities are removed. The cancer cells in the sample are then adsorbed onto a solid phase carrier, and the adsorbed cancer cells are collected. With this procedure, it is possible to recover live colon cancer cells released in the stool efficiently.

In addition, the present invention provides a method of screening cancer cells in stool. The method of the present invention comprises the steps of: (i) selecting a group of genes satisfying that (1) expression of the selected genes is observed in a live normal cell, that (2) expression of the selected genes is observed in a live cancer cell and that (3) expression of the selected genes is not observed in a dead cell; and (ii) screening cancer cells by analyzing expression of the group of selected genes in stool without separating cancer cells from normal cells.

The present invention is based on the finding that among cells collected from stool, normal cells are mostly dead while cancer cells are surviving (Matsushita, H. et al., Gastroenterology 129, 1918-1927 (2005)). Thus, if a set of genes are selected such that expression of the selected genes is observed in live normal and cancer cells and are not observed in dead cells, then it is possible to detect cancer cells without separating them from normal cells by analyzing expression of those genes in cells collected from stool. It is further possible to provide a set of genes and a method capable of screening the presence or absence of cancer cells even when the sample contains blood, by selecting such genes that expression of the genes is not observed in peripheral blood.

The present invention provides a group of 84 genes listed in Table 37. The present invention also provides a method for screening cancer cells from cells collected from stool by analyzing and comparing the gene expression amounts of cells in stool samples of healthy subjects and cancer subjects, for at least two genes selected from the group of genes.

It is found, as a result of gene expression analysis of stool using a variety of genes, that ribosomal protein genes show a high expression level in a gene expression analysis of stool.

The genes listed in Table 28 and Table 30 are genes which are particularly suitable for screening for the presence or absence of small amounts of colon cancer cells released in stool.

Thus, early stage diagnosis of colon cancer can be performed with high precision if expression of selected genes is analyzed for cells collected from stool according to the present invention.

With the above colon cancer cell screening, examination and diagnosis of colon cancer, particularly of early colon cancer, can be made easily. The present invention also provides an examination method for colon cancer in vitro.

Furthermore, the present invention also provides a primer for amplifying specifically any one of the genes listed in Table 1 and Table 30, a probe specifically hybridizing with any one of the genes listed in Table 1 and Table 30 for detection of the gene, and an immobilized sample in which the probe is immobilized on a solid-phase carrier. These primers, probes, and/or immobilized samples can be used in examination for colon cancer as a gene detection kit for the genes listed in Table 1 and Table 30.

The present invention provides a gene set (gene marker set) for colon cancer testing of at least two or more genes selected from the 50 genes listed in Table 1, and a gene set for colon cancer testing of at least two or more genes selected from the 84 genes listed in Table 37. The present invention also provides primers, probes and immobilized samples for analyzing the expression of these genes. According to the present invention, because the expression of genes can be simultaneously analyzed in the sample, early diagnosis of colon cancer is easily carried out.

Further features of the present invention will become apparent from the following description of exemplary embodiments with reference to the attached drawings.

### BRIEF DESCRIPTION OF THE DRAWINGS

FIG. 1 shows results of RT-PCR of RNAs of blood and surgically resected colon cancer tissues for selected genes.

FIG. 2 shows results of RT-PCR of RNAs collected from stool samples.

FIG. 3 shows chip hybridization results of RNAs collected from stool samples.

### DESCRIPTION OF THE EMBODIMENTS

The present invention is described below in detail. Any description is one example of implementing the present invention, and they by no means restricts the present invention.

With the present invention, in order to provide information useful for diagnosing colon cancer, 50 types of genes which were judged to have a high amount of expression in colon cancer tissue but no or extremely low expression in normal tunica mucosa coli were selected through various expression analyses.

With a commercial microarray, the expression profiles for approximately 39000 genes were obtained for early colon cancer tissue, advanced colon cancer tissue, and normal tunica mucosa coli. Using these results and a public database, 50 genes were selected (Table 1). Of these, there were 30 genes whose expression was not detected in colon mucosa or peripheral blood and which were strongly expressed in early colon cancer (Dukes' A, B) and which is still expressed in advanced colon cancer (Dukes' C, D). There were 16 genes which were strongly expressed in advanced colon cancer and whose expression was still detected in early colon cancer. There were 4 genes which were expressed strongly in all stages of cancers (Dukes' A-D).

Furthermore, through RT-PCR, 15 genes which were positive for one or both of early colon cancer (22 cases of mixed samples) and advanced colon cancer (B cases of mixed samples) and which were confirmed to be negative in peripheral blood was selected (Table 26). These 15 types of genes were negative even with nested PCR (high cycle PCR conducted twice with 2 sets of primers). These are genes which can differentiate between hemorrhoids and colon cancer. In other words, by using expression of these genes as an indictor, screening for presence or absence of cancer cells can be conducted even if blood is contained in the sample.

In order to provide information useful for diagnosis of colon cancer, the inventors have selected genes by using a number of expression analysis methods such that expression of the genes is observed at a high level in cells collected from stool samples of colon cancer subjects while it is observed at a null or very low level in cells collected from stool samples of healthy subjects. The group of genes of the present invention is selected by utilizing the finding, that among cells collected from stool, normal cells are mostly dead and only cancer cells are surviving. In other words, the group of genes of the present invention is selected such that expression of the genes is observed in a live cell and not, observed in a dead cell.

Using microarrays which are commercially available, expression profiles of about 39,000 genes were obtained. Then, 84 genes were selected based on the profiles and the public databases (Table 37). The 84 genes listed in Table 37 are a group of genes whose expression is observed at a high level in cells collected from stool samples of colon cancer subjects and not observed in cells collected from stool samples of healthy subjects.

Among the group of genes listed in Table 37, genes are further selected such that expression of the genes is not observed in peripheral blood (Table 38). The presence or absence of cancer cells can be detected based on these genes even when the collected sample contains blood. It is therefore possible to reduce the rate of false positives due to hemorrhoids, which has been a difficult problem in colon cancer testing.

The genes selected as mentioned above highly include ribosomal protein genes (Table 39). Generally, expression of ribosomal protein genes is proportional to the growth rate of a cell. The findings that growth rates of cancer cells are particularly high while normal cells are mostly dead in stool have lead to the inventive idea of the present invention that it is useful to effect colon cancer screening based on the analysis of expression of ribosomal protein genes using stool samples. Thus, ribosomal protein genes other than those as listed above may possibly be candidates for useful genes in screening cancer cells.

Since the amount of sample RNA extracted from stool is very small (submicrogram level), a step of amplifying is usually needed to examine expression of genes. As a method of amplification, in vitro transcription is generally used. After the amplification step, the presence or absence of expression of genes selected from the set of genes listed in Table 37, Table 38 or Table 39 can be determined by the RT-PCR method or like methods. As a method for examining expression of a large number of genes, DNA microarrays can be used advantageously.

In addition, with cells obtained from the stool of colon cancer subjects and healthy subjects, analysis with a commercial microarray and RT-PCR were conducted, and 7 genes (Table 30) that can be used to screen particularly for the presence or absence of cancer cells were selected. The genes listed in Table 28 and Table 30 are extremely good for screening for cancer cells in cells obtained from stool.

The genes selected in the present invention have a strong association with colon cancer cells. As a result, by measuring the expression levels of these genes, screening for colon cancer cells can be conducted. In particular, the gene expression profile which is measured using the probe of the present invention can be used for early diagnosis of colon cancer.

With the method of the present invention, the expression levels of each gene are measured with high sensitivity through fluorescent intensity or radiation intensity of the labeled probe. With regard to measurements, the appropriate standardization is conducted for each probe and each sample. By conducting a prognostication which can be compared between samples, a more accurate determination is possible. For example, when there is a difference in the amount of RNA recovered for each sample, by comparing the expression amount of the target gene with the expression amount of genes which have a constant expression amount such as housekeeping genes (for example beta actin), adjustments to the recovery amount are possible.

For the screening method of the present invention, colon cancer screening is conducted using a gene set described in Table 1, Table 26, Table 28, Table 30 or Table 37. Screening is conducted by measuring the expression amount of 2 or more genes selected from the gene set. The expression amount of the genes can be measured by measuring the mRNA amount in the sample or it can be detected by using immunostaining or ELISA of the protein which is the gene product.

For the genes listed in Tables 1, 26, 28, and 30, examples of a suitable base sequence for a probe for specifically detecting each gene is indicated by SEQ ID NOs: 1-50 and 151-157. Each probe contains the partial sequence for the base sequence of each gene. However, the probes are made so that non-specific hybridization with partial sequences from other genes is prevented as much as possible. The probes all have a chain length of 50-60 mer base length. With the assumption that there will be simultaneous hybridization with a plurality of probes, the probes are adjusted so that there is not a large variability among the probes in Tm values and the like. However, as long as the desired effect is not lost, looking at the base sequence of the target gene, the base length can be adjusted as suitable. In addition, as long as the specificity to the corresponding gene is not lost, each of the probes can be designed to have a base sequence in which 1 or more bases are deleted, substituted, or added with respect to the base sequences indicated in SEQ ID NOs: 1-50 and 151-157.

In addition, with the nucleic acids extracted from cells obtained from the sample (human stool, for example), for example, DNA can be hybridized with the probe as a double stranded DNA. As a result, the sequence for the probe used in such a situation can be the complementary sequence to the base sequence shown in Table 1. With the screening method of the present invention, two or more of these probes are used as a set. In other words, the present invention provides a probe set which can detect 2 or more types of genes selected from the genes listed in Table 1. Furthermore, the present invention provides a probe set which can detect 2 or more genes selected from genes listed in Table 30.

The probes of the present invention are highly specific, and they have a strict one-to-one correspondence with the target gene. As a result, there is no cross-hybridization, and a plurality of types of probes can be used simultaneously. The probe of the present invention can be used in the liquid phase or solid phase, but from the standpoint of simultaneous detection of a plurality of genes, preferably, each probe is immobilized on a carrier which is physically separated.

When using in the solid phase, the method for immobilization of the probe is not limited. Known immobilization methods such as adsorption, ionic bonding, covalent bonding and the like can be used as appropriate. In this situation, in order to have a stronger bond, as long as there is no significant loss of hybridization between sample and probe, there can be chemical modification of the probe, and the bonding can take advantage of the modification residue. Examples of such a chemical modification include methods for introducing an amino group to the 5′ terminus or methods for introducing a thiol group or methods for modifying with biotin.

The form of the solid-phase carrier is not particularly limited and can be a flat substrate, beads, fibers, and the like. In addition, the material is not limited and can be metal, glass, polymer, or the like.

A suitable example of an immobilized sample includes a DNA microarray in which a plurality of genes can be detected simultaneously with high sensitivity. A DNA microarray is a device for detecting nucleic acids in which a plurality of probes are arranged in a dense array on the surface of a flat substrate. Various known methods can be used to create DNA microarrays. In one example, glass is used as the solid-phase carrier. This glass is treated with an amino silane coupling agent which introduces an amino group. After further introducing a maleimido group onto the surface through EMCS or the like, an oligonucleotide probe which has been modified with a thiol group on the 5′ terminus reacts with the maleimido group. The probe is brought to the glass surface through a covalent bond.

When the carrier is a flat carrier such as a glass substrate, pipetting and the like is a representative means for supplying the probe onto the surface of the carrier. However, in order to supply a smaller amount of probe solution at high density, liquid supplying methods using ink jet methods such as bubble jet method or piezo method are used.

For the screening method of the present invention, there are two main pre-treatments which are conducted on the sample. These pre-treatments are labeling for detection and amplification to improve sensitivity. However, labeling is not always necessary if, after hybridization with the probe, there is a separate means for detecting hybridization of the probe with the sample. In addition, if the detection target is present in the sample in large quantities, amplification is not always needed.

Because in general there is only a small quantity of sample RNA (submicrogram), an amplification step is usually necessary. For the amplification method, in vitro transcription reaction or RT-PCR reaction is generally used. With amplification by RT-PCR, for the region to be amplified, in other words in the nucleic acid base sequence of each gene, the two primers which surround the region set by the probe must be set accurately. For each gene listed in Table 1, Table 26, Table 28, and Table 30 selected for the present invention, primers which can specifically amplify these genes were designed. An example of a suitable base sequence for each primer is indicated by SEQ ID NOs: 51-150 and 158-171. As with the probe, with the primer, it is assumed that there will be simultaneous amplification of a plurality of genes. The primers are adjusted so that there are no large variations in Tm values and the like among the primers. However, as long as the desired specificity and amplification rate is not diminished, the base sequence can be added or subtracted. For the addition to or subtraction from the base sequence, one or several bases is added or subtracted from the 5′ terminus or 3′ terminus or from both.

The labeling of the sample RNA is easily implemented by using a labeled substrate in the amplification step described above. Alternatively, there is a method in which the primer itself is labeled in advance. There is also a method in which, after the amplification step, a labeling substance is chemically or enzymatically bonded to a prescribed functional group of the sample. Labeling methods include known labeling methods such as fluorescent labeling, radiolabeling, enzymatic labeling and the like.

In the PCR reaction, the primers of the present invention have a high specificity with respect to the genes. Several types can be used in combination. A combined primer sets can be used in RT-PCR with the sample RNA as a template.

In addition, by combining these primer sets with an immobilized sample as described above, for example the DNA microarray, this can be used as a kit to detect specific genes. Of course, even just the primer set diluted in a suitable buffer solution can be used as a gene detection kit.

The probe, primer, immobilized sample, as well as the gene detection method of the present invention can be used for colon cancer diagnosis as described above. However, even with different objectives or samples, the present invention can be used for detecting genes described in Table 1, Table 26, Table 28, Table 30 and Table 37.

The screening of colon cancer cells is conducted by analysis of genes (mRNA) as described above. In addition, the present invention can be implemented by analyzing the expression amount of the proteins which are the translation product of the genes. The analysis of the expression amount of the proteins which are the gene products is implemented by known methods, such as western blotting method, dot blotting, slot blotting method, ELISA method, and RIA method, using antibody specific to the protein.

### EXAMPLES

Below, we describe the present invention in further detail by showing concrete embodiments.

### Example 1

### Selection Step 1

### Primary Selection of Marker Genes for Colon Cancer Screening

(1) Total RNA Extraction

Peripheral blood, 6 cases of normal tunica mucosa coli, 6 cases of early colon cancer tissue (Dukes' A, B) and 19 cases of advanced colon cancer tissue (Dukes' C, D) were collected, and total RNA was recovered. Recovery of total RNA was conducted according to the usual methods, and the following method was conducted.

First, each tissue sample was crushed (peripheral blood was used as it is), and ISOGEN from Nippon Gene Co. was added, and this was homogenized. A small amount of chloroform was added. This was centrifuged at 8000 rpm for 15 minutes. The supernatant was collected, and an equal amount of isopropanol to the collection amount was added. This was incubated for 15 minutes or longer at room temperature. This was centrifuged for 15 minutes at 15000 rpm, and the pellet was collected. Then, with ethanol precipitation (70%), the total RNA was obtained.

(2) Obtaining the Expression Profiles of About 39000 Genes by Microarray, and Selection of Marker Genes

In the stool of colon cancer patients, living cells other than bacteria include a small amount of cancer cells, lymphocytes, red blood cells and anal squamous cells. It is presumed that the cells shed from the tunica mucosa coli do not include living cells. In contrast, in the stool of healthy subjects (including those with hemorrhoids), there are lymphocytes, red blood cells and anal squamous cells. Therefore, genes that are expressed in almost all cases of early and advanced colon cancer and that are not expressed in peripheral blood and in squamous cells are potentially good markers for screening of colon cancer from stool. By taking into consideration that there could be very small amounts of living cells from shedding of the tunica mucosa coli, there was an additional condition that the gene not be expressed in the normal tunica mucosa coli, and the number of marker candidates was narrowed. The narrowing was conducted using a genome-wide gene expression analysis using a microarray.

For the microarray, human U133 oligonucleotide probe arrays (Affymetrix, US) were used according to the method recommended by the manufacturer. This will be described briefly. From the 5 μg of total RNA, a cDNA having a T7 RNA polymerase promoter was synthesized. Next, a biotinylated cRNA probe was created by the T7-transcription method. Next, 10 μg of the chemically cleaved cRNA was reacted with the microarray at 45° C. for 16 hours. The array was cleaned with 6×SSPE at 25° C. This was further cleaned with a secondary cleaning solution (100 mM MES (pH 6.7), 0.1 M NaCl, and 0.01% Tween 20) at 50° C. Next, the re-associated molecules were stained with streptavidin phycoerythrin (MolecularProbes) and then washed with 6×SSPE. This was further reacted with biotinylated anti-streptavidin IgG and re-stained with streptavidin phycoerythrin and then cleaned with 6×SSPE. The signal on the microarray was read by GeneArray scanner (made by Affymetrix) at a resolution of 3 μm. The intensities were analyzed using computer software Microarray Suite 5.0 (made by Affymetrix).

Gene expression amounts were analyzed using Microsoft Excel. As a result of selecting genes that were detected in all colon cancer cases but that were not detected in normal tunica mucosa coli and in peripheral blood, 50 types of genes were selected (Table 1). As a result of surveying the 50 genes in a public database (SBM DB: http://www.lsbm.org/db/index.html), these genes were found to have extremely low expression in skin and in squamous cells of the uterine cervix. Therefore, all of these 50 genes had satisfied the requirements that the inventors considered for markers for colon cancer screening. For these 50 genes, specific probes and primers were designed as shown in Table 1 and Table 2.

TABLE 1

| | | | | |
|---|---|---|---|---|
| No | Gene name | GenBank ID | Probe (5′→3′) | Sequence ID No. |

| | | | | |
|---|---|---|---|---|
| 1 | PAP | NM_002560 | TTCCCCCAACCTGACCACCTCATTCTTATCTTTCTTCTGTTTCTTCCTCCCCGCTGTCAT | SEQ ID NO: 1 |
| 2 | REG1A | AF172331 | GACCATCTCTCCAACTCAACTCAACCTGGACACTCTCTTCTCTGCTGAGTTTGCCTTGTT | SEQ ID NO: 2 |
| 3 | COL1A1 | Y15916 | GAGGCATGTCTGGTTCGGCGAGAGCATGACCGATGGATTCCAGTTCGAGTATG | SEQ ID NO: 3 |
| 4 | MMP11 | NM_005940 | CATGACAACTGCCGGGAGGGCCACGCAGGTCGTGGTCACCTGCCAGCGACTGTCTC | SEQ ID NO: 4 |
| 5 | SERPIN85 | NM_002639 | CAGGGGCTTCTAGCTGACTCGCACAGGGATTCTCACAATAGCCGATATCAGAATTTGTGT | SEQ ID NO: 5 |
| 6 | DPEP1 | NM_004413 | CAGATGCCAGGAGCCCTGCTGCCCACATGCAAGGACCAGCATCTCCTGAGAG | SEQ ID NO: 6 |
| 7 | DEFA5 | NM_021010 | TATTGCCGAACCGGCCGTTGTGCTACCCGTGAGTCCCTCTCCGGGGTGTGTGAAAT | SEQ ID NO: 7 |
| 8 | TACSTD2 | NM_002353 | ATCTGTATGACAACCCGGGATCGTTTGCAAGTAACTGAATCCATTGCGACATTGTGAAGG | SEQ ID NO: 8 |
| 9 | MMP7 | NM_002423 | ACAGGATCGTATCATATACTCGAGACTTACCGCATATTACAGTGGATCGATTAGTGTCAA | SEQ ID NO: 9 |
| 10 | SLCO4A1 | NM_016354 | CAGCATTCCTGCACTAACGGCAACTCTACGATGTGTCCGTGACCCTCAGAGATC | SEQ ID NO: 10 |
| 11 | SFRP4 | NM_003014 | ACAAACCCGAAAAGAGTGTGAGCTAACTAGTTTCCAAAGCGGAGACTTCCGACTTCCTTA | SEQ ID NO: 11 |
| 12 | COL11A1 | J04177 | ACTTGCACGTGTCCCTGAATTCCGCTGACTCTAATTTATGAGGATGCCGAACTCTGATGG | SEQ ID NO: 12 |
| 13 | KRT23 | NM_015515 | GGAACTGACGCAGCTACGCCATGAACTGGAGCGGCAGAACAATGAATACCAAG | SEQ ID NO: 13 |
| 14 | RAB2 | NM_002865 | CCGCGGCCATGGCGTACGCCTATCTCTTCAAGTACATCATAATCGGCGACACA | SEQ ID NO: 14 |
| 15 | KIAA1199 | NM_018689 | TCTGTTGCCGAAATAGCTGGTCCTTTTTCGGGAGTTAGATGTATAGAGTGTTTGTATGTA | SEQ ID NO: 15 |
| 16 | MCM7 | NM_005916 | CAGGACCGGCCCGACCGAGACAATGACCTACGGTTGGCCCAGCACATCACCTAT | SEQ ID NO: 16 |
| 17 | LY6G6D | NM_021246 | GTGCGCTGTGCTAGGTCAGCACCACAACTACCAGAACTGGAGGGTGTACGAC | SEQ ID NO: 17 |
| 18 | G3BP | NM_005754 | GAAGAAGACTCGAGCTGCCAGGGAAGGCGACCGACGAGATAATCGCCTTCGG | SEQ ID NO: 18 |
| 19 | ABHD4 | NM_022060 | CACTGGCCGAGGATAAGCCCGTCCCTGTCCCACATTCTAGCCCCACTATGCG | SEQ ID NO: 19 |
| 20 | POLD2 | NM_006230 | ACTGCAGCGTATCAAACTAAAAGGCACCATTGACGTGTCAAAGCTGGTTACGGGGACTGT | SEQ ID NO: 20 |
| 21 | TIF1 | NM_003852 | TAATACCACTACCCGTGAATTATATGGCCTGACAATATGAATTAGGTGTACTGTACTGAA | SEQ ID NO: 21 |
| 22 | CYP2B6 | NM_000767 | TAGTCTTCCCCAGTCCTCATTCCCAGCTGCCTCTTCCTACTGCTTCCGTCTATCAAAAAG | SEQ ID NO: 22 |
| 23 | TNK1 | AF097738 | TGGCCACATGGGACCAAGCGGAACCAGAACAAGGTCCCGACAGGGGTAGACGTT | SEQ ID NO: 23 |
| 24 | HSPH1 | NM_006644 | AGTCAAAGTGCGAGTCAACACCCATGGCATTTTCACCATCTCTACGGCATCTATGGTGGA | SEQ ID NO: 24 |
| 25 | NQO1 | NM_000903 | GTCTTAGAACCTCAACTGACATATAGCATTGGGCACACTCCAGCAGACGCCCGAATTCAA | SEQ ID NO: 25 |
| 26 | IRO039700 | BC006214 | TGGGCCCAAGGCTCATGCACACGCTACCTATTGTGGCACGGAGAGTAAGGAC | SEQ ID NO: 26 |
| 27 | FLJ10535 | NM_018129 | CCACCACGCTTGGCCGGGATAGTATATTTTTATAGCACTTCCCCTACTGATTGCTGCCTT | SEQ ID NO: 27 |
| 28 | SLC5A1 | NM_000343 | GAAATATTGCGGTACCAAGGTTGGCTGTACCAACATCGCCTATCCAACCTTAGTGGTGGA | SEQ ID NO: 28 |
| 29 | SYNCRIP | NM_006372 | ATGACGATTACTACTATTATGGTCCACCTCATATGCCCCCTCCAACAAGAGGTCGAGGGC | SEQ ID NO: 29 |
| 30 | AURKB | BC080581 | TATGTCTGTGTATGTATAGGGGAAAGAAGGGATCCCTAACTGTTCCCTTATCTGTTTTCT | SEQ ID NO: 30 |
| 31 | DDX17 | NM_006386 | CTCTGCAAGCTATCGGGATCGTAGTGAAACCGATAGAGCTGGTTATGCTAATGGCAGTGG | SEQ ID NO: 31 |
| 32 | HSPA4 | BC002526 | GCCGGCGGCATCGAGACTATCGCTAATGAGTATAGCGACCGCTGCACGCCGG | SEQ ID NO: 32 |
| 33 | SCN10A | NM_006514 | AAAGGCCTATCGGAGCTATGTGCTGCACCGCTCCATGGCACTCTCTAACACC | SEQ ID NO: 33 |
| 34 | IgH VH | AB035175 | AAGAACACGCTGTATCTGCAAATGCACAGGCTGAGAGCCGAGGACACGGCCGTATA | SEQ ID NO: 34 |
| 35 | MTAP | AF109294 | GCCCGGCGATATTGTCATTATTGATCAGTTCATTGACAGGACTATTTGCCACGACATTTC | SEQ ID NO: 35 |
| 36 | NEK3 | NM_002498 | CCCTCACATCGCCCCTCGGCTACAACGCTTCTCTCTCGAGGCATCGTAGCTCG | SEQ ID NO: 36 |
| 37 | ITGB4 | NM_000213 | GGAGGACTACGACAGCTTCCTTATGTACAGCGATGACGTTCTACGCTCTCCATCGG | SEQ ID NO: 37 |
| 38 | MET | NM_000245 | CTGCCTGACCTTTAAAAGGCCATCGATATTCTTTGCTCCTTGCCATAGGACTTGTATTGT | SEQ ID NO: 38 |
| 39 | KPNA6 | NM_012316 | CCTTTGTTAACACTCCTTACCAAGTCCACACGACTGACGATGACACGGAATGCAGTCTGG | SEQ ID NO: 39 |
| 40 | PROX1 | NM_002763 | CAGCACCGCCGAAGGGCTCTCCTTGTCGCTCATAAAGTCCGAGTGCGGCGATCTTCAAGA | SEQ ID NO: 40 |
| 41 | WTAP | NM_004906 | GGAAGTTTACGCCTGATAGCCAAACAGGGAAAAAGTTAATGGCGAAGTGTCGAATGCTTA | SEQ ID NO: 41 |
| 42 | FLJ10858 | NM_018248 | AAAGGCCGGATGCTAGGTGATGTGCTAATGGATCAGAACGTATTGCCTGGAGTAGGGAAG | SEQ ID NO: 42 |
| 43 | PHF16 | NM_014735 | AGCCCAGTTGTAGTAGGTGCCAGTCAGTCAAGGCAGGGGCCCTCTCTCCGTCAATA | SEQ ID NO: 43 |
| 44 | TRE5 | X78262 | CATGTTGGCCAAGCTAGTCTCGAACTACTGACTTCGGGTGATCTGCCCTCCTCG | SEQ ID NO: 44 |
| 45 | ICI_CGAP_Ut | M27830 | TCGCCCGTCACGCACCGCACGTTCGTGGGGAACCTGGCGCTAAACCATTCGTA | SEQ ID NO: 45 |
| 46 | TRIM31 | AF230386 | CTCAGGATACGAAGACATTTGACGTTGCGCTGTCCGAGGAGCTCCATGCGGCAC | SEQ ID NO: 46 |
| 47 | AP1S1 | NM_001283 | GCTGATCCACCGATACGTGGAGCTCTTAGACAAATACTTTGGCAGTGTGTGCGAGCTGGA | SEQ ID NO: 47 |
| 48 | CYLC2 | NM_001340 | CTCTCAAACCAACTCGTACTGTCGAGGTGGATTCTAAAGCAGCAGAAATTGGTAAGAAAG | SEQ ID NO: 46 |
| 49 | DHX9 | BF313832 | CTCAGAAACGGACGACGCAAGAAGTGCAAGCGACTTCTAGAATTCAGAACCGAAAGTGGA | SEQ ID NO: 49 |
| 50 | REG1B | NM_006507 | AACTGGTCCTGCAATTACTATGAAGTCAAAAATTAAACTAGACTATGTCTCCAACTCAGT | SEQ ID NO: 50 |

TABLE 2

| | | | | | |
|---|---|---|---|---|---|
| No. | Gene name | Forward-Primer(5′→3′) | Sequence ID No. | Reverse-Primer(5′→3′) | Sequence ID No. |

| | | | | | |
|---|---|---|---|---|---|
| 1 | PAP | GAGAAGCACAGCATTTCTGAG | SEQ ID NO: 51 | TGCTCTTTAAAGCCTTAGGCC | SEQ ID NO: 52 |
| 2 | REG1A | AATCCTGGCTACTGTGTGAG | SEQ ID NO: 53 | TCCAAAGACTGGGGTAGGT | SEQ ID NO: 54 |
| 3 | COL1A1 | CTACTACCGGGCTGATGATG | SEQ ID NO: 55 | GGAGGACTTGGTGGTTTTGT | SEQ ID NO: 56 |
| 4 | MMP11 | CACGAATATCAGGCTAGAGAC | SEQ ID NO: 57 | CACATTTACAATGGCTTTGGAG | SEQ ID NO: 58 |
| 5 | SERPINB5 | GGCTCCAGTGAAACTTGG | SEQ ID NO: 59 | CAAGGTAACGTGAGCACTT | SEQ ID NO: 60 |
| 6 | DPEP1 | ACCCATTACGGCTACTCCTC | SEQ ID NO: 61 | AAGGGGTGTTGCTTTTATTGC | SEQ ID NO: 62 |
| 7 | DEFA5 | CAGCCATGAGGACCATC | SEQ ID NO: 63 | TAGAAAGACACAAGGTACACA | SEQ ID NO: 64 |
| 8 | TACSTD2 | GAGAAAGGAACCGAGCTTGT | SEQ ID NO: 65 | TGGTAGTAAGGGCAAGCTGA | SEQ ID NO: 66 |
| 9 | MMP7 | TGGCCTACCTATAACTGGAA | SEQ ID NO: 67 | AATGGATGTTCTGCCTGAAG | SEQ ID NO: 68 |
| 10 | SLCO4A1 | GAAGGCCACCTGAACCTAAC | SEQ ID NO: 69 | CCATCTGAAGACTCCGACAG | SEQ ID NO: 70 |
| 11 | SFRP4 | GATCTTCAAGTCCTCATCACC | SEQ ID NO: 71 | ACCAGCTTTAACTCACCTTC | SEQ ID NO: 72 |
| 12 | COL11A1 | GTACGTCCAGAAAAGGCTATG | SEQ ID NO: 73 | GGACTTAGGGTCATCGGAA | SEQ ID NO: 74 |
| 13 | KRT23 | AGGTGACATCCACGAACT | SEQ ID NO: 75 | GTAGGAAAGTAGAGCTTTACCC | SEQ ID NO: 76 |
| 14 | RAB2 | CAGTTCGTCCGGCTTCCTC | SEQ ID NO: 77 | ATGAAGATGAGTCCATGTTCTCGT | SEQ ID NO: 78 |
| 15 | KIAA1199 | ACTGCACCCATGAGACT | SEQ ID NO: 79 | GTTCATGGTGATGCCTACAA | SEQ ID NO: 80 |
| 16 | MCM7 | GTGGAGAACTGACCTTAGAG | SEQ ID NO: 81 | TCCTTTGACATCTCCATTAGC | SEQ ID NO: 82 |
| 17 | LY6G6D | CTCCTCCTGTTCCTATGTG | SEQ ID NO: 83 | GCAGGAGAAGCATCGATG | SEQ ID NO: 84 |
| 18 | G3BP | AGAGTGCGAGAACAACGAAT | SEQ ID NO: 85 | ACTAAAGGTCAGGAAAGGGAA | SEQ ID NO: 86 |
| 19 | ABHD4 | CTTCACCTGCAGAGTCCTTT | SEQ ID NO: 87 | GATAGAGGGTCATGCAGTGG | SEQ ID NO: 88 |
| 20 | POLD2 | CAACTCCTCACAACCCTTCC | SEQ ID NO: 89 | TTCTTGGTGAGGTATTTGGC | SEQ ID NO: 90 |
| 21 | TIF1 | GAAGAAACGCCTCAAAAGC | SEQ ID NO: 91 | TTTTCTTTAATACAGTTGCCATCT | SEQ ID NO: 92 |
| 22 | CYP2B6 | CAAGCTGTCACTCCCCATAC | SEQ ID NO: 93 | GGGAGGTCAGGCTTTAGAGA | SEQ ID NO: 94 |
| 23 | TNK1 | TGGTTTCTGCCATCCGGAA | SEQ ID NO: 95 | CTTGATCCTCTCTAGCGCGTAA | SEQ ID NO: 96 |
| 24 | HSPH1 | CAATACTTTCCCCGGCAT | SEQ ID NO: 97 | CCTAACTGCCAGACCAAG | SEQ ID NO: 98 |
| 25 | NQO1 | CGCAGACCTTGTGATATTCC | SEQ ID NO: 99 | CGATTCCCTCTCATTTATTCCTT | SEQ ID NO: 100 |
| 26 | IRO039700 | GAGGATGATGAGCTGCTACA | SEQ ID NO: 101 | CTACACACTTTTATTGGAGGGG | SEQ ID NO: 102 |
| 27 | FLJ10535 | AACTCATTTACGGATAGGACTTT | SEQ ID NO: 103 | TTCCTGTCCTATTGGTACCA | SEQ ID NO: 104 |
| 28 | SLC5A1 | AAATGCTACACTCCAAGGGC | SEQ ID NO: 105 | CAGAAAATAGCAAGCAGGAAG | SEQ ID NO: 106 |
| 29 | SYNCRIP | AATGGGCTGATCCTATAGAAGA | SEQ ID NO: 107 | CTCTTTGTTGTTGGGCACCT | SEQ ID NO: 108 |
| 30 | AURKB | ACCTCATCTCCAAACTGCTCA | SEQ ID NO: 109 | AAAAAGCTTCAGCCTTTATTAAACA | SEQ ID NO: 110 |
| 31 | DDX17 | CTCAGAGGATTATGTGCACCG | SEQ ID NO: 111 | AGGAGGAGGAGGGTATTGGT | SEQ ID NO: 112 |
| 32 | HSPA4 | CAGTACCCACTGGAAGGACTTA | SEQ ID NO: 113 | TCTCCTTCAGTTTGGACAAAAG | SEQ ID NO: 114 |
| 33 | SCN10A | AGAACTTCAATGTGGCCACG | SEQ ID NO: 115 | CATACTGGTGGCTTCATCTT | SEQ ID NO: 116 |
| 34 | IgH VH | GTGGGTCTCAGCTATTAGTGG | SEQ ID NO: 117 | GGATTTCGCACAGTAATATACGG | SEQ ID NO: 118 |
| 35 | MTAP | CTCGCTTGGTTCCCTTAGTC | SEQ ID NO: 119 | ATCCCTGCAGGAAAAATCAT | SEQ ID NO: 120 |
| 36 | NEK3 | ATGTCAAGGGTGCATCAGTC | SEQ ID NO: 121 | GAACCCTTCTGAACCTGGTC | SEQ ID NO: 122 |
| 37 | ITGB4 | TCTGGCCTTCAATGTCGTCT | SEQ ID NO: 123 | TGTGGTCGAGTGTGAGTGTT | SEQ ID NO: 124 |
| 38 | MET | ATGTCCATGTGAACGCTACT | SEQ ID NO: 125 | CCAAGCCTCTGGTTCTGATG | SEQ ID NO: 126 |
| 39 | KPNA6 | CAAGAGTGGTGGATCGGTTC | SEQ ID NO: 127 | TCAACAAGTGGAGAAGGCAA | SEQ ID NO: 128 |
| 40 | PROX1 | TGATGGCCTATCCATTTCAG | SEQ ID NO: 129 | AACATCTTTGCCTGCGATAA | SEQ ID NO: 130 |
| 41 | WTAP | AAGCAACAACAGCAGGAGTC | SEQ ID NO: 131 | TGTGAAATCCAGACCCAGAC | SEQ ID NO: 132 |
| 42 | FLJ10858 | ATTTCGGAATGAAAGGCTTC | SEQ ID NO: 133 | CCCTGCTAGATGTCCAACTG | SEQ ID NO: 134 |
| 43 | PHF16 | GCTTATACCCTGTTCCCAAA | SEQ ID NO: 135 | ACACAGCTCCATCATAATTTCAT | SEQ ID NO: 136 |
| 44 | TRE5 | GCTCAGTGCTAGTCTGTTGTGTAG | SEQ ID NO: 137 | CCCTGGCCTCAAGTGATCC | SEQ ID NO: 138 |
| 45 | CI_CGAP_Ut | GAATTCACCAAGCGTTGGA | SEQ ID NO: 139 | GCTGACTTTCAATAGATCGCAG | SEQ ID NO: 140 |
| 46 | TRIM31 | TGTTCCTCTGGAACTGGAGA | SEQ ID NO: 141 | CCCTCCTTTTGCTCAAGAAT | SEQ ID NO: 142 |
| 47 | AP1S1 | GAAAGCCCAAGATGTGCAG | SEQ ID NO: 143 | GAGTCCCACCAAGAACAGG | SEQ ID NO: 144 |
| 48 | CYLC2 | AGAGTAAACTTTGGGCCATATGA | SEQ ID NO: 145 | ACCTTTTTTGCTATCTTTCTCTGT | SEQ ID NO: 146 |
| 49 | DHX9 | CTTGAATCATGGGTGACGTT | SEQ ID NO: 147 | GTTTGGTGCGATTATGTGGT | SEQ ID NO: 148 |
| 50 | REG1B | CTCAGGATTCAAGAAATGGAAGG | SEQ ID NO: 149 | GTGAAGGTACTGAAGATCAGCG | SEQ ID NO: 150 |

### Example 2

### Optimization of the PCR Conditions for the 50 Selected Genes

(1) Reverse Transcription (First Strand Synthesis)

With 5 μg of the total RNA of the advanced colon cancer tissue obtained in Example 1, reverse transcription was conducted with a random hexamer primer using SuperScript Choice System from Invitrogen. The following is a more concrete description of the method.

The total RNA was adjusted to a concentration of 10 μg/10 μl. To this, 1 μl of random hexamer primer was added. This was heat denatured by incubation at 68° C. for 10 minutes. This was rapidly cooled by placing on ice for 2 minutes or greater. Next, the reagents shown in Table 3 were added. This was incubated for 25° C. for 10 minutes, 42° C. for 60 minutes and 68° C. for 15 minutes. Afterwards, this was cooled rapidly and after spinning down, 1 μl of RNase H was added, and this was maintained at 37° C. for 20 minutes. In this way, approximately 20 μl of 1st strand cDNA solution was recovered.

TABLE 3

| Reagent | Amount to be added |
|---|---|
| 5x 1st strand buffer | 4 μl |
| 10 mM dNTP | 1 μl |
| 0.1 M DTT | 2 μl |
| RNase inhibiter | 0.5 μl |
| SuperScript II RT | 1 μl |

(2) PCR Amplification

Using the recovered 1st strand cDNA solution as a template, PCR amplification of the 50 genes described in Table 1 was conducted. For the template, in each PCR reaction, a 3 times dilution of the 1st strand cDNA solution of the colon cancer tissue synthesized in (1) was used. For the primer, the primer set described in Table 2 was used. As the PCR reaction solution, the PCR enzyme Takara Ex Taq from Takara Bio was used, and the solution was prepared with the composition shown in Table 4.

TABLE 4
Reaction mixture composition

| Reagent | Amount to be added | |
|---|---|---|
| Takara Ex Taq | 0.5 | μl (2.5 U) |
| 10x Ex Buffer (20 mM Mg²⁺) | 5.0 | μl |
| Template cDNA | 1.0 | μl |
| Forward Primer (F) (10 μM) | 2.5 | μl (25 pmol/tube) |
| Reverse Primer (R) (10 μM) | 2.5 | μl (25 pmol/tube) |
| dNTP Mix | 2.5 | μl (200 μlM) |
| Distilled water | 36.0 | μl |
| Total | 50 | μl |

For the reaction solution that was prepared, PCR amplification reaction was conducted using a commercially available thermocycler according to the temperature cycle protocol of Table 5. After completion, the reaction solution was stored at 4° C.

TABLE 5
Temperature condition for PCR amplification

| Step | Temperature | | Holding time | Repeat No. |
|---|---|---|---|---|
| 1 | 95° C. | | 5 min. | |
| 2 | 95° C. | (denaturation) | 30 sec. | 30 cycles |
| 3 | 58° C. | (annealing) | 30 sec. | |
| 4 | 72° C. | (extension) | 40 sec. | |
| 5 | 72° C. | | 10 min. | |

(3) Electrophoresis

Using 10 μl from each of the resulting PCR products, 1.5% agarose gel electrophoresis was conducted, and this was stained with EtBr solution. As a result, for each PCR product, a single thick band at a desired chain length was detected, hence it was clearly shown that there was one main product.

As seen in the experiments of (1) through (3) described above, by extracting the total RNAs from colon cancer cells according to the usual methods, and by conducting RT-PCR using the designed primers, amplification of the targeted amplification region was certainly performed.

### Example 3

### Selection Step 2

### Secondary Selection by RT-PCR of the 50 Genes

Cells separated from stool by MACS (magnetic cell sorting) which uses epithelial cell specific antibody (Dynabeads Epithelial Enrich, Invitrogen International) (this is described in detail in 1 of Example 6) hardly contain any lymphocytes or red blood cells. As a result, the 50 genes selected in the selection step 1 are genes that are effective for colon cancer screening of these separated cells. On the other hand, in order to conduct screening by extracting RNA directly from stool, a further selection for genes which are not detected in lymphocytes and red blood cells is needed.

As with Example 1, total RNA was extracted from another 22 cases of Dukes' A, B and 8 cases of Dukes' C, D and from peripheral blood. In order to understand the characteristics of the 50 genes, RT-PCR was conducted by the following method.

(1) Reverse Transcription Reaction (Synthesis of Single Stranded cDNA)

Using SuperScript Choice System from Invitrogen, reverse transcription of 5 μg of the total RNA with a random hexamer primer was conducted. Stated more concretely, the following method was used.

The total RNA was adjusted to a concentration of 10 μg/10 μl. To this, 1 μl of random hexamer primer was added. This was heat denatured by incubating at 68° C. for 10 minutes. After rapid cooling by placing on ice for 2 minutes or greater, the reagents shown above in Table 3 were added. This was incubated at 25° C. for 10 minutes, 42° C. for 60 minutes, and 68° C. for 15 minutes. Afterwards, this was rapidly cooled and spun down. Next, 1 μl of RNase H was added, and this was maintained at 37° C. for 20 minutes. With this method, approximately 20 μl of 1st strand cDNA solution was recovered.

(2) PCR Amplification

Using the recovered 1st strand cDNA solution as a template, PCR amplification of the 50 genes listed in Table 1 was conducted. For the template, in each PCR reaction, a three times dilution of the 1st strand cDNA solution of the colon cancer cell from (1) was used. For the primer, the primer set described in Table 2 was used.

For the PCR reaction, PCR kit Takara Ex Taq from Takara Bio was used, and the reaction solution shown above in Table 4 was prepared. For the prepared reaction solution, a commercially available thermocycler was used. The PCR amplification reaction was conducted according to the temperature cycle protocol of the above Table 5. After completing the PCR, the reaction solution was stored at 4° C.

(3) Electrophoresis

Using 10 μl of each of the resulting PCR product, 1.5% agarose gel electrophoresis was conducted, and this was stained with EtBr solution.

(4) Experiment Results

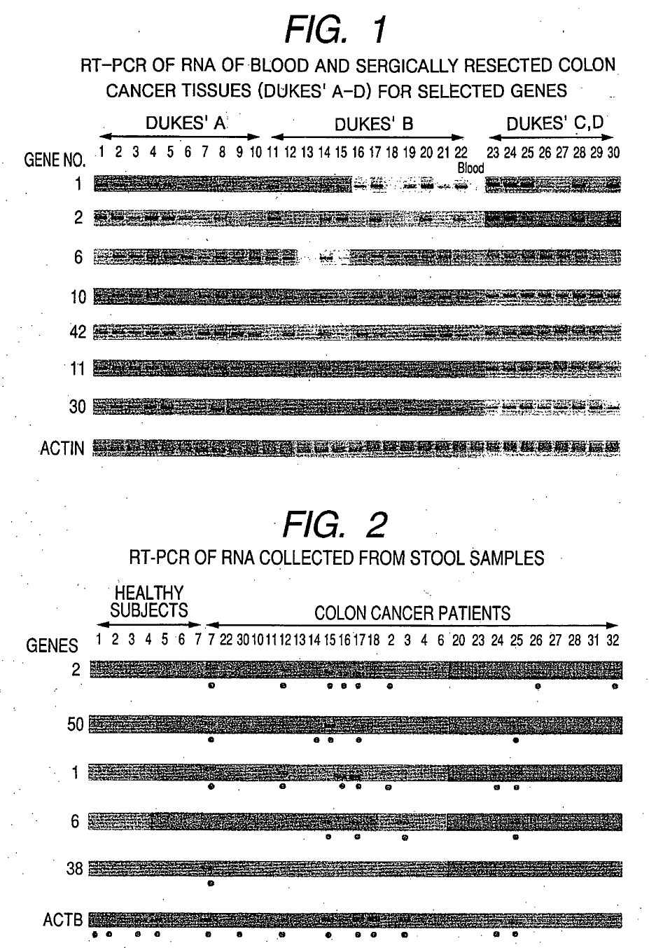

Of the 50 genes, there were 15 genes which were not detected in peripheral blood (genes No. 1, 2, 6, 8, 10, 11, 25, 30, 37, 38, 40, 41, 42, 46, 50). Of these, electrophoresis results of representative genes (No. 1, 2, 6, 10, 11, 30, and 42) are shown in FIG. 1. In addition, in Table 6, the presence or absence of expression of all 15 genes in the 30 cases of colon cancer tissue is shown. In both early cancer (Dukes' A, B) and advanced cancer (Dukes' C, D), expression was observed in 70-100% of cases. In all cases, expression of a plurality (7-15) of genes was observed.

These 15 genes were not detected in peripheral blood after 30 cycles of PCR, and even with a further 20 cycles. From these results, we concluded that these were marker genes which can differentiate colon cancer from hemorrhoids. The 15 genes described above and their probes and primers are summarized as shown in Tables 26 and 27.

TABLE 6
Expression of the 15 genes not detected in the blood in 30 cases of colon cancer tissues

| | 15 genes | | | | | | | | | | | | | | | |
|---|---|---|---|---|---|---|---|---|---|---|---|---|---|---|---|---|
| Samples | 1 | 2 | 6 | 10 | 42 | 11 | 30 | 8 | 25 | 37 | 38 | 40 | 41 | 46 | 50 | |
| Dukes' A | 90 | 90 | 100 | 90 | 90 | 100 | 100 | 100 | 100 | 90 | 100 | 100 | 100 | 90 | 100 | % |
| (n = 10) | | | | | | | | | | | | | | | | |
| Dukes' B | 83 | 83 | 92 | 100 | 100 | 83 | 100 | 100 | 100 | 92 | 100 | 100 | 92 | 42 | 100 | % |
| (n = 12) | | | | | | | | | | | | | | | | |
| Dukes' C, D | 75 | 88 | 100 | 100 | 100 | 100 | 100 | 100 | 100 | 100 | 100 | 100 | 100 | 88 | 100 | % |
| (n = 8) | | | | | | | | | | | | | | | | |
| Total | 83 | 87 | 97 | 97 | 97 | 93 | 100 | 100 | 100 | 93 | 100 | 100 | 97 | 70 | 100 | % |
| (n = 30) | | | | | | | | | | | | | | | | |
| Blood | (—) | (—) | (—) | (—) | (—) | (—) | (—) | (—) | (—) | (—) | (—) | (—) | (—) | (—) | (—) | |
| (58 mix) | | | | | | | | | | | | | | | | |

### Example 4

### Expression Analysis by DNA Microarray

I. Preparation of DNA Microarray

(1) Cleaning of Glass Substrate

A synthetic quartz glass substrate (size (WxLxT): 25 mm×75 mm×1 mm, Iiyama Precision Glass) was placed in a heat-resistant and alkali-resistant rack. A cleaning solution for ultrasonic cleaning was prepared at a prescribed concentration, and the glass substrate was immersed in this cleaning solution. After immersing in the cleaning solution overnight, ultrasonic cleaning was conducted for 20 minutes. Next, the glass substrate was taken out, then rinsed lightly with pure water, and subjected to ultrasonic cleaning with ultrapure water for 20 minutes. Thereafter, the glass substrate was immersed for 10 minutes in 1 N sodium hydroxide solution which was heated to 80° C. and again cleaned with pure water and ultrapure water. Thus, a cleaned quartz glass substrate for use in DNA chip was prepared.

(2) Surface Treatment

A silane coupling agent KBM-603 (made by Shin-Etsu Chemical) was dissolved in pure water to achieve a concentration of 1%. This was stirred for 2 hours at room temperature. Subsequently, the cleaned quartz glass substrate was immersed in the silane coupling agent solution, and this was left for 20 minutes at room temperature. The glass substrate was pulled out, and after cleaning the surface lightly with pure water, both surfaces of the glass substrate were dried by blowing nitrogen gas. Next, the glass substrate dried by nitrogen blowing was baked for 1 hour in an oven heated to 120° C., and the coupling agent treatment was completed. By this coupling agent treatment, amino groups derived from the silane coupling agent was introduced onto the glass substrate surface.

An EMCS solution was prepared by dissolving N-(6-Maleimidocaproyloxy)succinimide (abbreviated as EMCS) made by DOJINDO in a 1:1 mixture solvent of dimethylsulfoxide and ethanol to achieve a final concentration of 0.3 mg/ml. After completion of the baking, the coupling agent treated glass substrate was cooled and then was immersed for two hours in the EMCS solution at room temperature. During this immersion treatment, the amino group, which was introduced onto the glass substrate surface by the coupling agent treatment, reacted with the succinimide group of EMCS, and the maleimide group from EMCS was introduced onto the surface of the glass substrate. The glass substrate was pulled out of the EMCS solution and was washed using the mixture solvent of dimethylsulfoxide and ethanol described previously. This was further cleaned with ethanol and then dried under a nitrogen gas atmosphere.

(3) Synthesis of Probe DNA

Each of the probe DNA (SEQ ID NOs: 1-50) for detecting the 50 genes shown in Table 1 was synthesized.

In order to have a covalent bond between the probe DNA and the maleimido group which was introduced onto the glass substrate as described above, thiol treatment of the 5′ terminus of the probe DNA was performed according to the standard method. Afterwards, in order to avoid side reactions during DNA synthesis, the protective group was deprotected, and further HPLC purification and desalting treatment were performed. Each of the resulting probe DNA was dissolved in pure water and aliquoted so that the final concentration (when dissolved in the ink) would be 10 μM. Freeze-drying was then conducted to remove the water content.

(4) Probe DNA Ejection by BJ Printer and Bonding to the Substrate Surface

An aqueous solution containing 7.5 wt % glycerin, 7.5 wt % thiodiglycol, 7.5 wt % urea, 1.0 wt % acetylenol EH (made by Kawaken Fine Chemicals) was prepared. Next, the aliquoted probe DNA was dissolved in this mixture solvent to a prescribed concentration (10 μM). An ink tank for a bubble jet printer (product name: BJF-850 by Canon), was filled with the resulting probe DNA solution, and this was attached to the printer head.

The bubble jet printer was modified to accommodate ink jet printing onto a flat surface. In addition, with this modified bubble jet printer, by inputting a printing pattern according to a prescribed file creation method, DNA solution droplets of approximately 5 μl can be spotted at a pitch of approximately 190 μm.

Next, using the modified bubble jet printer, spotting operation of the probe DNA solution onto the glass substrate surface was performed. A printing pattern was created beforehand so that 16 spots would be ejected for each probe onto one DNA microarray. Thus, ink jet printing was conducted. Using a magnifying glass or the like, spotting of the DNA solution in the desired pattern was confirmed. Next, this was placed in a humidified chamber for 30 minutes at normal temperature. The maleimido group of the glass substrate surface and the sulfanyl (—SH) group on the 5′ terminus of the probe DNA were reacted.

(5) Cleaning

After reacting for 30 minutes in the humidified chamber, any unreacted probe DNA remaining on the glass substrate surface was washed away with 100 mM NaCl containing 10 mM phosphate buffer solution (pH 7.0). A DNA microarray was obtained in which the prescribed single stranded probe DNA was fixed onto the glass substrate surface at 16 spots per DNA chip.

II. Hybridization Reaction

(1) Amplification of Sample and Labeling (PCR Amplification with Incorporation of Label)

For the sample, the 1st strand cDNA solution synthesized in Example 1 was used. Of 50 genes in Table 1, for 10 of these genes which are shown in Table 9, PCR amplification with label incorporation was conducted using the 1st strand cDNA as the template. For the primer, the primer set shown in Table 2 was used. The PCR reaction was conducted by preparing the reaction solution shown in Table 7 using the PCR enzyme Takara Ex Taq made by Takara Bio. The solution was prepared so that the final concentration of dNTP was 200 μM.

TABLE 7
Reaction mixture composition

| Reagent | Amount to be added | |
|---|---|---|
| Takara Ex Taq | 0.5 | μl (1.0 U) |
| 10x Ex Taq Buffer (20 mM Mg²⁺) | 5.0 | μl |
| Template DNA (cDNA solution) | 1.0 | μl |
| Forward Primer (F) (10 μM) | 2.5 | μl (25 pmol/tube) |
| Reverse Primer (R) (10 μM) | 2.5 | μl (25 pmol/tube) |
| dNTP Mixture (*) | 2.0 | μl |
| Cy3 dUTP (1.0 mM, Amersham Biosciences) | 2.0 | μl (40 μM) |
| Distilled water | 34.5 | μl |
| Total | 50 | μl |

(*) Concentration: 5.0 mM for dATP, dCTP and dGTP; 4.0 mM for dTTP

With regard to the prepared reaction solution, a commercially available thermocycler was used to conduct PCR amplification reaction according to the temperature cycle protocol seen in Table 5. After completion, the reaction solution was stored at 4° C.

After the reaction was completed, the reaction solution was purified with a purification column (Qiagen Co. QIAquick PCR Purification Kit). After eluting with 50 μl of distilled water, the resulting purified product was the labeled sample.

Using the DNA microarray created in I and the 10 labeled samples, hybridization was conducted on the microarray.

(2) Blocking of the DNA Microarray

BSA (bovine serum albumin Fraction V: made by Sigma) was dissolved in 100 mM NaCl/10 mM Phosphate Buffer to a concentration of 1 wt %. The DNA microarray prepared in II was immersed in this solution for 2 hours at room temperature, and blocking of the glass substrate surface was conducted. After blocking was completed, the DNA microarray was cleaned with a 0.1×SSC solution (15 mM of NaCl, 1.5 mM of sodium citrate (trisodium citrate dihydrate C₆H₅Na₃-2H₂O), pH 7.0) containing 0.1 wt % SDS (sodium dodecyl sulfate). Next, this was rinsed with pure water. Next, the DNA microarray was dewatered with a spin dry apparatus.

(3) Preparation of Hybridization Solution

The hybridization solution was prepared for each PCR product so that the final concentration was 6×SSPE/10% Formamide/PCR amplification product solution (6×SSPE: 900 mM of NaCl, 60 mM of NaH₂PO₄—H₂O, 6 mM of EDTA, pH 7.4). For each PCR amplification product solution, 25.0 μl, which is approximately half of the purified product, was used.

(4). Hybridization

The dewatered DNA chip was set on a hybridization apparatus (Hybridization Station from Genomic Solutions Inc.). Using the hybridization solution with the above described composition, the hybridization reaction was conducted with the procedure and conditions shown in Table 8.

TABLE 8
Conditions and procedures for hybridization

| Operation | Operation procedures and conditions |
|---|---|
| Reaction | 65° C. 3 min → 92° C. 2 min → 55° C. 4 h |
| Washing | 2x SSC/0.1% SDS at 25° C. |
| | 2x SSC at 20° C. |
| (Rinse) | Distilled water (manual rinse washing) |
| Drying | Spin dry |

(5) Fluorescence Measurement

After completion of the hybridization reaction, the fluorescence of the hybrid on the DNA chip that had been spun dry was measured using DNA microarray fluorescence detection apparatus (Genepix 4000B made by Axon). The results for the measured fluoroluminance are shown in Table 9.

For the calculation of luminance, first, the actual measured value of the fluorescent intensity was calculated by subtracting a background value from the apparent fluorescent intensity from each spot. Then, the fluoroluminance value was calculated as an average value for the 16 spots. The background value was the fluorescent intensity that was seen on the DNA chip in areas where there was no probe DNA spot.

As is clear from these results, the expression of each gene has an adequate signal and can be measured. Similar experiments were conducted with other genes. With all of the probes and primers, gene expression analysis that is specific and highly sensitive was possible.

TABLE 9

| No. | Gene name | Fluoroluminescence |
|---|---|---|
| 1 | PAP | 1286.9 |
| 2 | REGIA | 1089.3 |
| 6 | DPEP1 | 286.2 |
| 10 | SLCO4A1 | 5814.5 |
| 30 | STK12 | 271.9 |
| 37 | ITGB4 | 3729.6 |
| 38 | MET | 8321.3 |
| 41 | WTAP | 1181.2 |
| 42 | FLJ10858 | 1921.1 |
| 46 | TR1M31 | 1162.9 |

### Example 5

### Expression Analysis with the DNA Microarray of the Gene which has been Amplified by Multiplex RT-PCR

(1) Amplification and Labeling of the Sample (Multiplex PCR Amplification with Label Incorporation)

For the samples, colon cancer tissue from advanced colon cancer, normal tunica mucosa coli, and blood were collected. With regard to each sample, the total RNA was recovered and synthesis of the 1st strand was conducted by the procedure indicated in Examples 1 and 2. In order to eliminate individual differences, the samples collected from 7 patients were mixed. With the resulting 1st strand cDNA solution as a sample, gene amplification and labeling reaction are shown below. In the present example, the 10 genes which have been selected in Example 4 were the targets. With regard to the primers, all 10 types of primers are added to one PCR tube. In other words, multiplex PCR was conducted. For the substrate, Cy3-dUTP was added as in Example 4, and labeling of the PCR product was conducted. The solution composition of the PCR reaction is as shown in Table 10.

TABLE 10
Reaction mixture composition

| Reagent | Amount to be added | |
|---|---|---|
| Takara Ex Taq | 2.5 | μl (2.5 U) |
| 10x Ex Taq Buffer (20 mM Mg²⁺) | 5.0 | μl |
| Template DNA (cDNA solution) | 1.0 | μl |
| Forward Primer (F) | 12.5 | pmol/each |
| Reverse Primer (R) | 12.5 | pmol/each |
| dNTP Mixture (*) | 2.0 | μl |
| Cy3 dUTP (1.0 mM, Amersham Biosciences) | 2.0 | μl (40 μM) |
| Distilled water | Optional | |
| Total | 50 | μl |

(*) Concentrations are the same as shown in Table 7.

For the reaction solution that was prepared, a commercially available thermocycler was used to conduct PCR amplification reaction according to the temperature cycle protocol of Table 5 as described in Example 2. After completion, the reaction solution was stored at 4° C.

After the reaction was completed, the reaction solution was purified with a purification column (Qiagen Co. QIAquick PCR Purification Kit). After eluting with 50 μl of distilled water, the resulting purified product was the labeled sample. Using the DNA microarray created in I of Example 4 and these three types of labeled samples, hybridization was conducted on the microarray by the method indicated in Example 4. The fluoroluminance values for each probe are shown in Table 11.

TABLE 11

| | | Fluoroluminescence | | |
|---|---|---|---|---|
| | Gene | Colon | Normal | |
| No. | name | cancer | tissue | Blood |
| 1 | PAP | 516.5 | 47.1 | 0.0 |
| 2 | REG1A | 110.3 | 23.8 | 0.7 |
| 6 | DPEP1 | 152.9 | 132.2 | 6.5 |
| 10 | SLCO4A1 | 1025.1 | 236.2 | 0.0 |
| 30 | AURKB | 257.6 | 62.2 | 0.0 |
| 37 | ITGB4 | 2561.2 | 738.2 | 0.0 |
| 38 | MET | 5019.1 | 599.8 | 0.0 |
| 41 | WTAP | 1134.7 | 630.1 | 0.0 |
| 42 | FLJ10858 | 316.3 | 86.4 | 0.0 |
| 46 | TRIM31 | 88.0 | 0.0 | 0.0 |

As is clear from these results, even if the sample preparation is conducted by multiplex PCR, the expression for each gene has an adequate signal and can be measured.

In addition, each of the genes is expressed strongly in colon cancer and has a low expression amount in normal tissue. In blood, the genes were either hardly expressed at all, or there was a difference in the fluoroluminance value as compared to colon cancer cells. It was confirmed that even if blood is mixed in the sample, colon cancer diagnosis is still possible.

### Example 6

### RT-PCR Analysis Using the Cells Isolated from Stool of Colon Cancer Patients

RT-PCR analyses were carried out using RNA from cells isolated from stools of 7 normal subjects and 25 colon cancer patients for the 5 genes (No. 1, 2, 6, 38, and 50 that are PAP, REG1A, DPEP1, MET and REG1B, respectively). The 5 genes described above, and their probes and primers are shown altogether in Table 28 and 29.

(1) Isolation of Cells from Stool

Stool from colon cancer patients before operation was used as a sample. For using stool, we explained to patients details of the experiment beforehand and obtained the consent.

Two hundred ml of Hanks solution (Nissui, Nissui Pharmaceutical) containing 10% FBS was added to a stomacher bag containing stool (about 5-80 g), and after sealing, the stool suspension was prepared using a stomacher (200 rpm, 1 min).

When a stomacher bag with a filter was used, the suspension was filtered through the filter in the bag. When a stomacher bag without a filter was used, the suspension was filtered through a funnel type filter set on a cylinder shaped plastic container, and the filtrate was collected in a beaker. The filtrate was aliquoted to five 50 ml centrifuge tubes.

Forty μl of Ber-EP4 antibody bound magnetic beads (Dynabeads Epithelial Enrich, Invitrogen International) was added to each of the centrifuge tubes and stirred using a mix rotor (VMR-5, ASONE Co., Ltd.) (4° C., 60 rpm, 30 minutes) to bind cells in the filtrate to Ber-EP4 antibody.

Each of the centrifuge tubes was set to a magnetic stand (Dynal MPC-1, Invitrogen International), placed sideways on a mild mixer (SI-36, TAITEC Co., Ltd.) and moved in a seesaw-like motion for 15 minutes (60 rounds/minute) to stir the filtrate and to collect magnetic beads to the side wall of the centrifuge tube.

After removing the filtrate, the centrifuge tubes were taken out of the stand, and 500 μl of Hanks solution containing 10% FBS was added to each tube to wash the beads collected on the wall of the tube.

The wash solution containing the beads was recovered into 5 microtubes (1.5 ml, made by Eppendorf), each of which contained 500 μl of Hanks solution containing 10% FBS. The beads were suspended lightly, and then the microtubes were set on a magnetic stand (Dynal MPC-S, Invitrogen International) to collect the beads to the side wall of the microtube.

After removing the wash solution, microtubes were taken out of the stand, and 1 ml of Hanks solution containing 10% FBS was added to each tube and the beads collected on the wall of the microtubes were washed. Similarly, the tubes were set on the magnetic stand, the magnetic beads were collected on the side wall of the microtubes and pellets of cell-beads complex were obtained after removing the supernatant. Subsequently RNA was extracted from these pellets using ISOGEN (Nippon Genes).

(2) RT-PCR Analysis

(i) cDNA Synthesis (the First Round)

One μg of total RNA obtained as above was subjected to reverse transcription using oligo (dT) primer and SUPERSCRIPT Choice System (Invitrogen). Total RNA (10 μl) was mixed with 1 μl of 100 μM T7-oligo dT 24 primer (1 μg) and incubated at 65° C. for 10 minutes. Subsequently, the mixture was rapidly cooled by placing on ice for 2 minutes or longer, and then mixed with reagents shown in Table 12 and incubated at 37° C. for 2 minutes.

TABLE 12

| Reagent | Amount to be added |
|---|---|
| 5x 1st strand buffer | 4 μl |
| 10 mM dNTP | 1 μl |
| 0.1 M DTT | 2 μl |
| RNase inhibiter | 0.5 μl |

Then the reaction mixture was mixed with 1 μl of SuperScriptII RT and incubated at 37° C. for 1 hour. Thus, about 20 μl of the 1^{st} strand cDNA solution was recovered.

Next, the 2^{nd} strand cDNA was synthesized by the method described below. The 1^{st} strand cDNA solution was mixed with reagents as shown in Table 13 and incubated at 16° C. for 2 hours.

TABLE 13

| Reagent | Amount to be added |
|---|---|
| Distilled water | 91 μl |
| 5x 2nd strand buffer | 30 μl |
| 10 mM dNTP | 3 μl |
| E. coli DNA Ligase | 1 μl |
| E. coli DNA polymerase | 4 μl |
| E. coli RNase H | 1 μl |

Further, 2 μl of T4 DNA polymerase was added and incubated at 16° C. for 5 minutes to make the ends of the 2^{nd} strand cDNA smooth. Next, the 2^{nd} strand cDNA was purified. The product described above was mixed with reagents shown in Table 14 and centrifuged at 15,000 rpm for 10 minutes.

TABLE 14

| Reagent | Amount to be added |
|---|---|
| Glycogen (20 mg/ml) | 1 μl |
| Phenol | 150 μl |

Subsequently, 150 μl of chloroform was added, and the mixture was collected and centrifuged at 15,000 rpm for 10 min, and only the supernatant was collected and transferred to another tube. Further, reagents were added as shown in Table 15, and the mixture was stand at room temperature for 15 minutes, centrifuged at 15,000 rpm for 10 minutes, and only the supernatant was collected and transferred to another tube.

TABLE 15

| Reagent | Amount to be added |
|---|---|
| 7.5 M ammonium acetate aqueous | 75 μl |
| solution | |
| Isopropanol | 500 μl |

And then 500 μl of 70% ethanol was added and the mixture was centrifuged at 15,000 rpm for 10 minutes (ethanol rinse), and at this time the precipitates were kept and the solution was discarded. To the remaining precipitates, reagents were added as shown in Table 16 and the mixture was allowed to stand at room temperature for 15 minutes, centrifuged at 15,000 rpm for 10 minutes, and the solution was discarded while the precipitates were kept. To the remaining precipitates, 500 μl of 70% ethanol was added, the mixture was centrifuged at 15,000 rpm for 10 minutes and the solution was discarded. Finally the precipitates were air dried and dissolved in 8 μl of water.

TABLE 16

| Reagent | Amount to be added |
|---|---|
| Distilled water | 100 μl |
| 7.5 M ammonium acetate aqueous | 50 μl |
| solution | |
| Isopropanol | 300 μl |

(ii) Synthesis of cRNA: In Vitro Transcription (the First Round)

Following reaction was carried out using MEGAscript T7 kit (Ambion). To the 8 μl of cDNA solution prepared in (i), reagents were added as shown in Table 17 and the mixture was incubated at 37° C. for 5 hours. Next, 1 μl of DNase (RNase free) was added and the mixture was incubated at 37° C. for 15 minutes to remove DNA.

TABLE 17

| Reagent | Amount to be added |
|---|---|
| NTP mix | 8 μl |
| 10x T7 RNA polymerase Buffer | 2 μl |
| T7 RNA polymerase enzyme mix | 2 μl |

Subsequently, cRNA was purified. Reagents were added as shown in Table 18 and the mixture was centrifuged at 15,000 rpm for 5 minutes, further mixed with 300 μl of isopropanol, incubated at room temperature for 15 minutes, centrifuged at 15,000 rpm for 10 minutes, and only the supernatant was collected and transferred to another tube. Then the supernatant was mixed with 500 μl of 70% ethanol, centrifuged at 15,000 rpm for 5 minutes, and only the precipitates were kept, air dried and dissolved in 8 μl of water, while the solution was discarded.

TABLE 18

| Reagent | Amount to be added |
|---|---|
| Isogene (Nippon Gene) | 400 μl (5 fold dilution) |
| Chloroform | 100 μl |

(iii) cDNA Synthesis (2^{nd} Round)

The second reverse transcription reaction was carried out for cRNA solution obtained as above using random hexamer primers. One μl of 0.5 μg/μl random hexamer (1 μg) was added, and the mixture was incubated at 65° C. for 10 minutes. Then, after rapidly cooling by placing on ice for 2 minutes or longer, reagents were added as shown in Table 12 and the mixture was incubated at 37° C. for 2 minutes. Then, 1 μl of SuperScript II RT was added and the mixture was incubated at 37° C. for 1 hour. Thus, about 20 μl of the 1^{st} strand cDNA solution was recovered. Subsequently, RNA was removed by adding 1 μl of RNase H. The mixture was incubated at 37° C. for 20 minutes, then at 95° C. for 2 minutes to separate RNA and DNA and thereafter rapidly cooled by placing on ice for 2 minutes or longer.

Next, the 2^{nd} strand cDNA was synthesized by the method shown below. The 1^{st} strand cDNA solution was mixed with 1 μl of 100 μM T7-oligo dT 24 primer (1 μg) and incubated at 68° C. for 5 minutes and then at 42° C. for 10 minutes. Reagents were added as shown in Table 19, and the mixture was incubated at 16° C. for 2 hours. Further, 2 μl of T4 DNA polymerase was added and the mixture was incubated at 16° C. for 5 minute to make the ends of the 2^{nd} strand cDNA smooth. Next, the 2^{nd} strand cDNA was purified.

TABLE 19

| Reagent | Amount to be added |
|---|---|
| Distilled water | 91 μl |
| 5 × 2nd strand buffer | 30 μl |
| 10 mM dNTP | 3 μl |
| E. coli DNA polymerase | 4 μl |
| E. coli RNase H | 1 μl |

To the product described above, 150 μl of phenol was added and the mixture was centrifuged at 15,000 rpm for 10 minutes. Subsequently, 150 μl of chloroform was added, and the mixture was centrifuged at 15,000 rpm for 10 minutes, and only the supernatant was collected and transferred to another tube. Further reagents were added as shown in Table 15 and the mixture was incubated at room temperature for 15 minutes, centrifuged at 15,000 rpm for 10 minutes, and only the supernatant was collected and transferred to another tube. And then 500 μl of 70% ethanol was added and the mixture was centrifuged at 15,000 rpm for 10 minutes (ethanol rinse), and at this time only the precipitates were kept and the solution was discarded. To the remaining precipitates, reagents were added as shown in Table 16 and the mixture was allowed to stand at room temperature for 15 minutes, centrifuged at 15,000 rpm for 10 minutes, and the solution was discarded while the precipitates were kept. To the remaining precipitates, 500 μl of 70% ethanol was added, the mixture was centrifuged at 15,000 rpm for 10 minutes and the solution was discarded. Finally the precipitates were air dried and dissolved in 22 μl of distilled water.

(iv) cRNA Synthesis: In Vitro Transcription (Second Round)

cRNA was synthesized by the similar method used in (ii) from the 22 μl cDNA solution prepared in (iii). However, at the last step, RNA was dissolved in 1.0 μl of distilled water (RNA content was 5 μg-10 μg).

(v) Reverse Transcription Reaction (1^{st} Strand cDNA Synthesis)

One μl of 0.5 μg/μl random hexamer (1 μg) was added to 10 μl of cRNA solution prepared in (iv), and the mixture was incubated at 65° C. for 10 minutes. Then, after rapidly cooling the mixture by placing on ice for 2 minutes or longer, reagents were added as shown in Table 12 and the mixture was incubated at 37° C. for 2 minutes so that efficient reverse transcription reaction can be carried out. Then, 1 μl of SuperScript II RT was added and the mixture was incubated at 37° C. for 1 hour. Subsequently, RNA was removed by adding 1 μl of RNase H. The mixture was incubated at 37° C. for 20 minutes, then at 95° C. for 2 minutes to separate RNA and DNA and thereafter rapidly cooled by placing on ice for 2 minutes or longer. About 20 μl of the reaction solution described above was mixed with 20 μl of purified water, and 1 μl of the mixture was used as a template for PCR. The condition for PCR and electrophoresis of the products were similar to Example 3 (2) and (3).

(3) Experimental Results

All the stool samples from 7 healthy subjects gave negative results for the five genes, while the stool samples from 25 colon cancer patients were positive as a whole in about 50% ( 12/25) for at least one gene. For the cases in which actin mRNA was detected, indicating there were many cells, at least one gene was positive in about 80% ( 7/9) (FIG. 2). That is, the positive predictive value of this method is 100%, and is far superior to that of the occult blood test for stool (about 0.1%).

### Example 7

### High Sensitivity Chip Analysis Using Cells Isolated from Stool of Colon Cancer Patient

Expression analyses by DNA microarray were carried out for cDNA solution samples synthesized in Example 6 after multiplex PCR amplification by incorporated label. Similar to Example 6, 7 healthy subjects and 25 colon cancer patients, total 32 cases were analyzed.

In this Example, the five genes selected in Example 6 were targeted for detection, and multiplex PCR was carried out by adding all 5 kinds of primers to one PCR tube. Similar to Example 4, Cy3-dUTP was added as a substrate to label PCR products. The composition of PCR reaction mixture is shown in Table 20.

TABLE 20
Reaction mixture composition

| Component | Composition | |
|---|---|---|
| Invitrogen, AccuPrimer Taq | 1.0 | μl |
| 10x AccuPrime PCR Buffer | 2.5 | μl |
| Template DNA (cDNA solution) | 1.0 | μl |
| Forward Primer (F) | 6.25 | pmol/each |
| Reverse Primer (R) | 6.25 | pmol/each |
| Cy3 dUTP (1.0 mM, Amersham Biosciences) | 1.0 | μl (40 μM) |
| Distilled water | Optional | |
| Total | 25 | μl |

PCR amplification was carried out for prepared reaction mixtures using a commercially available thermal cycler according to the temperature cycle protocol of Table 4, described in Example 2. However, the cycle number was 35, and the reaction mixture was stored at 4° C. after the reaction.

After the reaction, the reaction product was purified using a purification column (QIAGEN, QIAquick PCR Purification Kit) and then made into labeled sample.

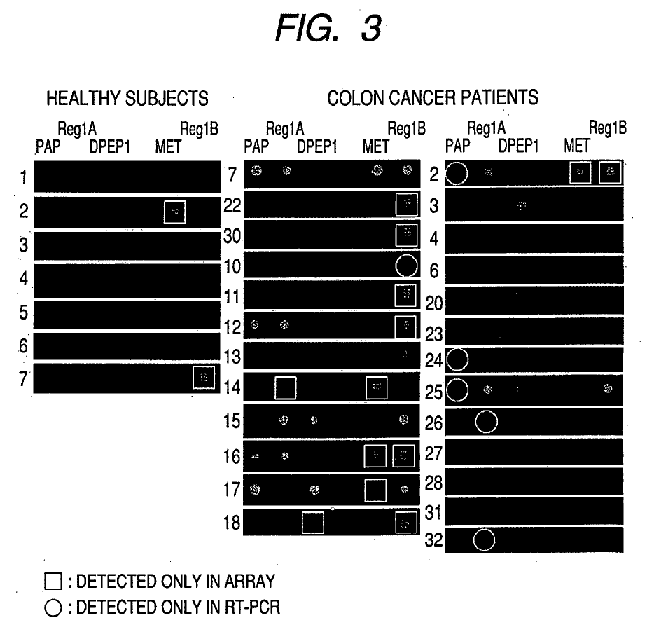

Hybridization on a microarray was carried out according to the method shown in Example 4 using a DNA microarray prepared in Example 4 I and these 32 kinds of labeled samples. The hybridization images obtained as a result are shown in FIG. 3 and fluoroluminescence values to each probe are also shown in Table 21.

TABLE 21

| | | No01 | No02 | No06 | No38 | No50 |
|---|---|---|---|---|---|---|
| | | PAP | PEG1A | DPEP1 | MET | REG1B |
| Healthy subject | 1 | 1.2 | 1.6 | 2.2 | 2.0 | 8.6 |
| | 2 | 1.0 | 0.6 | 0.4 | 74.6 | 1.8 |
| | 3 | 1.4 | 1.6 | 2.3 | 2.1 | 1.9 |
| | 4 | 1.9 | 1.1 | 1.8 | 1.4 | 19.8 |
| | 5 | 2.8 | 5.2 | 3.0 | 3.3 | 3.5 |
| | 6 | 0.9 | 0.4 | 1.2 | 1.7 | 1.9 |
| | 7 | 1.4 | 1.8 | 2.4 | 1.2 | 168.0 |
| Colon cancer patient | 7 | 4087.0 | 664.7 | 0.7 | 6165.6 | 2012.0 |
| | 22 | 11.2 | 6.7 | 2.2 | 1.3 | 202.9 |
| | 30 | 4.3 | 0.4 | 1.4 | 1.1 | 148.9 |
| | 10 | 2.3 | 2.9 | 3.1 | 2.6 | 2.5 |
| | 11 | 1.1 | 1.1 | 1.4 | 1.1 | 203.7 |
| | 12 | 496.2 | 678.1 | 2.2 | 0.6 | 81.0 |
| | 13 | 0.3 | 1.5 | 1.5 | 0.9 | 121.5 |
| | 14 | 0.0 | 29.3 | 0.0 | 107.6 | 31.2 |
| | 15 | 0.7 | 1069.3 | 510.9 | 0.0 | 2022.4 |
| | 16 | 267.4 | 469.9 | 7.3 | 88.9 | 234.6 |
| | 17 | 4342.1 | 34.1 | 2297.7 | 49.3 | 464.4 |
| | 18 | 1.6 | 1.0 | 48.8 | 1.7 | 159.8 |
| | 2 | 6.4 | 274.7 | 1.4 | 88.8 | 125.1 |
| | 3 | 0.6 | 0.8 | 153.7 | 1.1 | 1.0 |
| | 4 | 1.4 | 1.7 | 1.5 | 2.0 | 4.3 |
| | 6 | 2.7 | 2.1 | 2.7 | 3.3 | 2.4 |
| | 20 | 1.5 | 1.6 | 1.3 | 1.4 | 1.1 |
| | 23 | 1.5 | 1.9 | 2.2 | 1.3 | 1.5 |
| | 24 | 5.6 | 0.1 | 0.2 | 0.1 | 0.0 |
| | 25 | 0.4 | 680.6 | 83.7 | 0.3 | 841.6 |
| | 26 | 0.5 | 1.1 | 1.1 | 0.6 | 4.1 |
| | 27 | 0.2 | 0.0 | 0.0 | 1.2 | 0.6 |
| | 28 | 0.5 | 0.3 | 0.6 | 0.0 | 0.1 |
| | 31 | 1.0 | 1.0 | 0.9 | 1.6 | 1.9 |
| | 32 | 1.6 | 2.5 | 1.3 | 1.7 | 1.6 |

(3) Experimental Results

Table 22 summarizes the presence/absence of the expression of the 5 genes in each case. In fluoroluminescence values in Table 21, a value of 25 or above was defined as positive. If one gene or more among the 5 genes was positive, the judgment was “o”. The presence/absence of the expression of β-actin was based on the result of PCR in Example 6. Also, cytodiagnosis in the far right column of the table was the results of the cytodiagnosis for cells recovered from stool samples.

The results for the 5 genes indicated that one gene among the 5 genes was positive in 2 stool samples among 7 stool samples from healthy subjects (false positive 2/7). On the other hand, in 25 stool samples from colon cancer patients, the positivity rate was 56% ( 14/25) as a whole. For the cases in which actin mRNA was detected, indicating there were many cells, at least one gene was positive in about 90% ( 8/9). In the cytodiagnosis 6/25 were positive, and in comparison with this result, the result of the expression analysis using the gene set of the present invention would be significant data. Also the correct rate in the positive cases is high at about 90% ( 14/16), and is superior to that of the occult blood test for stool.

TABLE 22

| | | No01 | No02 | No06 | No38 | No50 | Positive | | |
|---|---|---|---|---|---|---|---|---|---|
| | | PAP | REG1A | DPEP1 | MET | REG1B | judgment | β-actin | Cytodiagnosis |
| Healthy subject | 1 | | | | | | | ◯ | |
| | 2 | | | | ◯ | | ◯ | ◯ | |
| | 3 | | | | | | | | |
| | 4 | | | | | | | ◯ | |
| | 5 | | | | | | | ◯ | |
| | 6 | | | | | | | | |
| | 7 | | | | | ◯ | ◯ | | |
| Colon cancer patients | 7 | ◯ | ◯ | | ◯ | ◯ | ◯ | ◯ | |
| | 22 | | | | | ◯ | ◯ | | |
| | 30 | | | | | ◯ | ◯ | ◯ | |
| | 10 | | | | | | | | ◯ |
| | 11 | | | | | ◯ | ◯ | | ◯ |
| | 12 | ◯ | ◯ | | | ◯ | ◯ | ◯ | ◯ |
| | 13 | | | | | ◯ | ◯ | | ◯ |
| | 14 | | ◯ | | ◯ | ◯ | ◯ | | ◯ |
| | 15 | | ◯ | ◯ | | ◯ | ◯ | ◯ | ◯ |
| | 16 | ◯ | ◯ | | ◯ | ◯ | ◯ | | |
| | 17 | ◯ | ◯ | ◯ | ◯ | ◯ | ◯ | ◯ | |
| | 18 | | | ◯ | | ◯ | ◯ | ◯ | |
| | 2 | | ◯ | | ◯ | ◯ | ◯ | | |
| | 3 | | | ◯ | | | ◯ | ◯ | |
| | 4 | | | | | | | | |
| | 6 | | | | | | | | |
| | 20 | | | | | | | | |
| | 23 | | | | | | | | |
| | 24 | | | | | | | ◯ | |
| | 25 | | ◯ | ◯ | | ◯ | ◯ | ◯ | |
| | 26 | | | | | | | | |
| | 27 | | | | | | | | |
| | 28 | | | | | | | | |
| | 31 | | | | | | | | |
| | 32 | | | | | | | | |

Comparison of the results of the cytodiagnosis with the expression analysis by microarray is shown in Table 23. Among the 6 cases of cytodiagnosis positive, cases are also detected by microarray. Further, among the 19 cases of cytodiagnosis negative, 9 cases, about 50%, can be diagnosed to be positive.

The results clearly demonstrated that colon cancer diagnosis can be possible using small amount of cells recovered from stool by preparing the sample by multiplex PCR and analyzing by microarray.

TABLE 23
Comparison with cytodiagnosis results

| | | | |
|---|---|---|---|
| Cancer patient | − (negative) | 9/19 (47%) | 14/25 (56%) |
| | + (positive) | 5/6 (83%) | |
| Healthy subject | | | 2/7 (28%) |

Further, the results of the analysis by the microarray of the present Example are superior in sensitivity compared to the results of RT-PCR shown in Example 6, and total 15 spots among the 32 samples were rescued. On the other hand, 6 spots could be detected by RT-PCR but not by microarray due to poor PCR amplification because it was multiplex PCR (FIG. 3). Combining the merits of the both methods, the presence/absence of the expression of the 5 genes in each case is summarized in Table 24. When the result of either RT-PCR or microarray was positive, the positive judgment “o” was given. Also, the positivity rate was calculated from this result and compared with the cytodiagnosis results (Table 25). By combining the detection results of RT-PCR and microarray as shown in Tables 24 and 25, the positivity rate of colon cancer patients was 72% ( 18/25) confirming that the gene set of the present invention is efficacious for the diagnosis of colon cancer.

TABLE 24

| | | No01 | | No02 | | No06 | | No38 | | No50 | | Positive | | |
|---|---|---|---|---|---|---|---|---|---|---|---|---|---|---|
| | | PAP | | REG1A | | DPEP1 | | MET | | REG1B | | judgment | β-actin | Cytodiagnosis |
| Healthy subject | 1 | | | | | | | | | | | | ◯ | |
| | 2 | | | | | | | | ◯ | | | ◯ | ◯ | |
| | 3 | | | | | | | | | | | | | |
| | 4 | | | | | | | | | | | | ◯ | |
| | 5 | | | | | | | | | | | | ◯ | |
| | 6 | | | | | | | | | | | | | |
| | 7 | | | | | | | | | | ◯ | ◯ | | |
| Colon cancer patient | 7 | ◯ | ◯ | ◯ | ◯ | | | ◯ | ◯ | ◯ | ◯ | ◯ | ◯ | |
| | 22 | | | | | | | | | | ◯ | ◯ | | |
| | 30 | | | | | | | | | | ◯ | ◯ | ◯ | |
| | 10 | | | | | | | | | ◯ | | ◯ | | ◯ |
| | 11 | | | | | | | | | | ◯ | ◯ | | ◯ |
| | 12 | ◯ | ◯ | ◯ | ◯ | | | | | | ◯ | ◯ | ◯ | ◯ |
| | 13 | | | | | | | | | | ◯ | ◯ | | ◯ |
| | 14 | | | | ◯ | | | | ◯ | ◯ | ◯ | ◯ | | ◯ |
| | 15 | | | ◯ | ◯ | ◯ | ◯ | | | ◯ | ◯ | ◯ | ◯ | ◯ |
| | 16 | ◯ | ◯ | ◯ | ◯ | | | | ◯ | | ◯ | ◯ | | |
| | 17 | ◯ | ◯ | ◯ | ◯ | ◯ | ◯ | | ◯ | ◯ | ◯ | ◯ | ◯ | |
| | 18 | | | | | | ◯ | | | | ◯ | ◯ | ◯ | |
| | 2 | ◯ | | ◯ | ◯ | | | | ◯ | | ◯ | ◯ | | |
| | 3 | | | | | ◯ | ◯ | | | | | ◯ | ◯ | |
| | 4 | | | | | | | | | | | | | |
| | 6 | | | | | | | | | | | | | |
| | 20 | | | | | | | | | | | | | |
| | 23 | | | | | | | | | | | | | |
| | 24 | ◯ | | | | | | | | | | ◯ | ◯ | |
| | 25 | ◯ | | | ◯ | ◯ | ◯ | | | ◯ | ◯ | ◯ | ◯ | |
| | 26 | | | ◯ | | | | | | | | ◯ | | |
| | 27 | | | | | | | | | | | | | |
| | 28 | | | | | | | | | | | | | |
| | 31 | | | | | | | | | | | | | |
| | 32 | | | ◯ | | | | | | | | ◯ | | |

TABLE 25
Comparison with cytodiagnosis results

| | | | |
|---|---|---|---|
| Cancer patient | − (negative) | 12/19 (63%) | 18/25 (72%) |
| | + (positive) | 6/6 (100%) | |
| Healthy subject | | | 2/7 (28%) |

TABLE 26

| | | | |
|---|---|---|---|
| No. | Gene name | probe (5′→3′) | Sequence ID No. |

| | | | |
|---|---|---|---|
| 1 | PAP | TTCCCCCAACCTGACCACCTCATTCTTATCTTTCTTCTGTTTTCTTCCTCCCCGCTGTCAT | SEQ ID NO: 1 |
| 2 | REG1A | GACCATCTCTCCAACTCAACTCAACCTGGACACTCTCTTCTCTGCTGAGTTTGCCTTGTT | SEQ ID NO: 2 |
| 6 | DPEP1 | CAGATGCCAGGAGCCCTGCTGCCCACATGCAAGGACCAGCATCTCCTGAGAG | SEQ ID NO: 6 |
| 8 | TACSTD2 | ATCTGTATGACAACCCGGGATCGTTTGCAAGTAACTGAATCCATTGCGACATTGTGAAGG | SEQ ID NO: 8 |
| 10 | SLCO4A1 | CAGCATTCCTGCACTAACGGCAACTCTACGATGTGTCCGTGACCCTCAGAGATC | SEQ ID NO: 10 |
| 11 | SFRP4 | ACAAACCCGAAAAGAGTGTGAGCTAACTAGTTTCCAAAGCGGAGACTTCCGACTTCCTTA | SEQ ID NO: 11 |
| 25 | NQO1 | GTCTTAGAACCTCAACTGACATATAGCATTGGGCACACTCCAGCAGACGCCCGAATTCAA | SEQ ID NO: 25 |
| 30 | AURKB | TATGTCTGTGTATGTATAGGGGAAAGAAGGGATCCCTAACTGTTCCCTTATCTGTTTTCT | SEQ ID NO: 30 |
| 3 | 8 MET | CTGCCTGACCTTTAAAAGGCCATCGATATTCTTTGCTCCTTGCCATAGGACTTGTATTGT | SEQ ID NO: 38 |
| 39 | KPNA6 | CCTTTGTTAACACTCCTTACCAAGTCCACACGACTGACGATGACACGGAATGCAGTCTGG | SEQ ID NO: 39 |
| 40 | PROX1 | CAGCACCGCCGAAGGGCTCTCCTTGTCGCTCATAAAGTCCGAGTGCGGCGATCTTCAAGA | SEQ ID NO: 40 |
| 41 | WTAP | GGAAGTTTACGCCTGATAGCCAAACAGGGAAAAAGTTAATGGCGAAGTGTCGAATGCTTA | SEQ ID NO: 41 |
| 42 | FLJ10858 | AAAGGCCGGATGCTAGGTGATGTGCTAATGGATCAGAACGTATTGCCTGGAGTAGGGAAC | SEQ ID NO: 42 |
| 46 | TRIM31 | CTCAGGATACGAAGACATTTGACGTTGCGCTGTCCGAGGAGCTCCATGCGGCAC | SEQ ID NO: 46 |
| 50 | REG1B | AACTGGTCCTGCAATTACTATGAAGTCAAAAATTAAACTAGACTATGTCTCCAACTCAGT | SEQ ID NO: 50 |

TABLE 27

| | | | | | |
|---|---|---|---|---|---|
| No. | Gene name | Forward-Primer(5′→3′) | Sequence ID No. | Reverse-Primer(5′→3′) | Sequence ID No. |

| | | | | | |
|---|---|---|---|---|---|
| 1 | PAP | GAGAAGCACAGCATTTCTGAG | SEQ ID NO: 51 | TGCTCTTTAAAGCCTTAGGCC | SEQ ID NO: 52 |
| 2 | REG1 | AAATCCTGGCTACTGTGTGAG | SEQ ID NO: 53 | TCCAAAGACTGGGGTAGGT | SEQ ID NO: 54 |
| 6 | DPEP1 | ACCCATTACGGCTACTCCTC | SEQ ID NO: 61 | AAGGGGTGTTGCTTTTATTGC | SEQ ID NO: 62 |
| 8 | TACSTD2 | GAGAAAGGAACCGAGCTTGT | SEQ ID NO: 65 | TGGTAGTAAGGGCAAGCTGA | SEQ ID NO: 66 |
| 10 | SLCO4A1 | GAAGGCCACCTGAACCTAAC | SEQ ID NO: 69 | CCATCTGAAGACTCCGACAG | SEQ ID NO: 70 |
| 11 | SFRP4 | GATCTTCAAGTCCTCATCACC | SEQ ID NO: 71 | ACCAGCTTTAACTCACCTTC | SEQ ID NO: 72 |
| 25 | NQO1 | CGCAGACCTTGTGATATTCC | SEQ ID NO: 99 | CGATTCCCTCTCATTTATTCCTT | SEQ ID NO: 100 |
| 30 | AURKB | ACCTCATCTCCAAACTGCTCA | SEQ ID NO: 109 | AAAAAGCTTCAGCCTTTATTAAACA | SEQ ID NO: 110 |
| 38 | MET | ATGTCCATGTGAACGCTACT | SEQ ID NO: 125 | CCAAGCCTCTGGTTCTGATG | SEQ ID NO: 126 |
| 39 | KPNA6 | CAAGAGTGGTGGATCGGTTC | SEQ ID NO: 127 | TCAACAAGTGGAGAAGGCAA | SEQ ID NO: 128 |
| 40 | PROX1 | TGATGGCCTATCCATTTCAG | SEQ ID NO: 129 | AACATCTTTGCCTGCGATAA | SEQ ID NO: 130 |
| 41 | WTAP | AAGCAACAACAGCAGGAGTC | SEQ ID NO: 131 | TGTGAAATCCAGACCCAGAC | SEQ ID NO: 132 |
| 42 | FLJ10858 | ATTTCGGAATGAAAGGCTTC | SEQ ID NO: 133 | CCCTGCTAGATGTCCAACTG | SEQ ID NO: 134 |
| 46 | TRIM31 | TGTTCCTCTGGAACTGGAGA | SEQ ID NO: 141 | CCCTCCTTTTGCTCAAGAAT | SEQ ID NO: 142 |
| 50 | REG1B | CTCAGGATTCAAGAAATGGAAGG | SEQ ID NO: 149 | GTGAAGGTACTGAAGATCAGCG | SEQ ID NO: 150 |

TABLE 28

| | | | |
|---|---|---|---|
| | Gene | | Sequence |
| No. | name | probe (5′→3′) | ID No. |

| | | | |
|---|---|---|---|
| 1 | PAP | TTCCCCCAACCTGACCACCTCATTCTT | SEQ ID NO: 1 |
| | | ATCTTTCTTCTGTTTCTTCCTCCCCGC | |
| | | TGTCAT | |
| 2 | REG1A | GACCATCTCTCCAACTCAACTCAACCT | SEQ ID NO: 2 |
| | | GGACACTCTCTTCTCTGCTGAGTTTGC | |
| | | CTTGTT | |
| 6 | DPEP1 | CAGATGCCAGGAGCCCTGCTGCCCACA | SEQ ID NO: 6 |
| | | TGCAAGGACCAGCATCTCCTGAGAG | |
| 38 | MET | CTGCCTGACCTTTAAAAGGCCATCGAT | SEQ ID NO: 38 |
| | | ATTCTTTGCTCCTTGCCATAGGACTTG | |
| | | TATTGT | |
| 50 | REG1B | AACTGGTCCTGCAATTACTATGAAGTC | SEQ ID NO: 50 |
| | | AAAAATTAACTAGACTATGTCTCCAAC | |
| | | TCAGT | |

TABLE 29

| | | | | | |
|---|---|---|---|---|---|
| No. | Gene name | Forward-Primer (5′→3′) | Sequence ID NO. | Reverse-Primer(5′→3′) | Sequence ID No. |

| | | | | | |
|---|---|---|---|---|---|
| 1 | PAP | GAGAAGCACAGCATTTCTGAG | SEQ ID NO: 51 | TGCTCTTTAAAGCCTTAGGCC | SEQ ID NO: 52 |
| 2 | REG1A | AATCCTGGCTACTGTGTGAG | SEQ ID NO: 53 | TCCAAAGACTGGGGTAGGT | SEQ ID NO: 54 |
| 6 | DPEP1 | ACCCATTACGGCTACTCCTC | SEQ ID NO: 61 | AAGGGGTGTTGCTTTTATTGC | SEQ ID NO: 62 |
| 38 | MET | ATGTCCATGTGAACGCTACT | SEQ ID NO: 125 | CCAAGCCTCTGGTTCTGAT | SEQ ID NO: 126 |
| 50 | REG1B | CTCAGGATTCAAGAAATGGAAGG | SEQ ID NO: 149 | GTGAAGGTACTGAAGATCAGCG | SEQ ID NO: 150 |

### Example 8

### Selection of the Marker Genes for Colon Cancer Screening Using Stool (Obtaining the Expression Profiles of About 39000 Genes by Microarray, and Selection of the Marker Genes)

Genome-wide gene expression analyses were carried out targeting the total RNA extracted in Example 6 (1) using a microarray (human U133 oligonucleotide probe arrays (Affymetric, USA)). Experiments were carried out according to the method recommended by the manufacturer. The targets used were 4 kinds of cell RNA isolated from stool samples of colon cancer patients and a mixture of 7 kinds of cell RNA isolated from stool samples of healthy subjects, total 5 kinds of RNA.

After synthesis of cDNA having a promoter of T7 RNA polymerase from 5 μg of the total RNA, biotinated cRNA probe was prepared by T7 transcription method. Ten micrograms of the cRNA is then chemically fragmented and reacted with a microarray at 45° C. for 16 hours. The array was washed with 6×SSPE at 25° C. and further washed with a secondary wash solution (100 mM MES at pH 6.7, 0.1 M NaCl, and 0.01% Tween 20) at 50° C. Subsequently, re-associated molecules were dyed with streptavidin phycoerythrin (Molecular Probes), then washed with 6×SSPE, further reacted with biotinylated anti-streptavidin IgG, again dyed with streptavidin phycoerythrin, and washed with 6×SSPE. Signals on the microarray were read using a GeneArray scanner (Affymetrix), and the intensity was analyzed using a computer software, Microarray Suite 5.0 (Affymetrix).

Amount of the gene expression was analyzed using Microsoft Excel, and 84 genes were chosen which were expressed at high level in all the cases of colon cancer described above but not detected in healthy subjects (Table 37). Further, 48 genes were chosen (Table 38) by excluding the genes detected in healthy subjects and in peripheral blood (result of Example 1). These 48 genes can determine the presence or absence of cancer cells even when the sample contains blood and therefore satisfy the requirements proposed by the inventors as a marker for screening of colon cancer using stool samples. Furthermore, 7 kinds of genes were chosen (Table 30) as ones expressed at a higher level. All of the 84 genes (including the 7 genes listed in Table 30) fulfilled the conditions for the screening markers for colon cancer using stool as samples, which were proposed by the present inventors; i.e., (1) expression in live normal cells is observed, (2) expression in cancer cells is observed, and (3) no expression is observed in dead cells. Specific probes and primers were designed as shown in Table 30 and Table 31 for these selected 7 genes.

TABLE 37

| | | | |
|---|---|---|---|
| 51 | SEPP1 | selenoprotein P, plasma, 1 | NM_005410 |
| 52 | RPL27A | ribosomal protein L27A | NM_000990 |
| 53 | ATP1B1 | ATPase, Na+/K+ transporting, | NM_001677 |
| | | beta 1 polypeptide | |
| 54 | EEF1A1 | eukaryotic translation | NM_001402 |
| | | elongation factor 1 alpha 1 | |
| 55 | SFN | stratifin | NM_006142 |
| 56 | RPS11 | ribosomal protein S11 | NM_001015 |
| 57 | RPL23 | ribosomal protein L23 | NM_000978 |
| 58 | JUND | jun D proto-oncogene | NM_005354 |
| 59 | TPT1 | tumor protein, | NM_003295 |
| | | translationally-controlled 1 | |
| 60 | RPL41 | ribosomal protein L41 | NM_021104 |
| 61 | RPS29 | ribosomal protein S29 | NM_001032 |
| 62 | RPL38 | ribosomal protein L38 | NM_000999 |
| 63 | B2M | beta-2-microglobulin | NM_004048 |
| 64 | CFL1 | cofilin 1 (non-muscle) | NM_005507 |
| 65 | RPL31 | ribosomal protein L31 | NM_000993 |
| 66 | RPS3A | ribosomal protein S3A | NM_001006 |
| 67 | TMSB10 | thymosin, beta 10 | NM021103 |
| 68 | RPL39 | ribosomal protein L39 | NM_001000 |
| 69 | HMGB1 | high-mobility group box 1 | NM_002128 |
| 70 | CEACAM6 | carcinoembryonic antigen- | NM002483 |
| | | related cell adhesion | |
| | | molecule 6 | |
| | | (non-specific cross reacting | |
| | | antigen) | |
| 71 | RPS20 | ribosomal protein S20 | NM_001023 |
| 72 | ARF6 | ADP-ribosylation factor 6 | NM_001663 |
| 73 | RPS21 | ribosomal protein S21 | NM_001024 |
| 74 | EIF5A | Eukaryotic translation | NM_001970 |
| | | initiation factor 5A | |
| 75 | RPL30 | ribosomal protein L30 | NM_000989 |
| 76 | RPL23A | ribosomal protein L23a | NM_000984 |
| 77 | LOC56902 | putatative 28 kDa protein | NM_010143 |
| 78 | RPL27 | ribosomal protein L27 | NM_000988 |
| 79 | CEACAM5 | carcinoembryonic antigen- | NM_004363 |
| | | related cell adhesion | |
| | | molecule 5 | |
| 80 | RPS24 | ribosomal protein S24 | NM_001026 |
| 81 | MARCKS | Myristoylated alanine-rich | NM_002356 |
| | | protein kinase C substrate | |
| 82 | PDE4C | phosphodiesterase 4C, cAMP- | NM_000923 |
| | | specific (phosphodiesterase | |
| | | E1sdunce homolog, Drosophila) | |
| 83 | LOC651423 | similar to mitogen-activated | XM_940575 |
| | | protein kinase kinase 3 | |
| | | isoform A | |
| 84 | RPS10 | ribosomal protein S10 | NM_001014 |
| 85 | CEP27 | centrosomal protein 27 kDa | NM_018097 |
| 86 | IL1RN | interleukin 1 receptor | NM_173842 |
| | | antagonist | |
| 87 | SLC35E1 | solute carrier family 35, | NM_024881 |
| | | member E1 | |
| 88 | RPS27 | ribosomal protein S27 | NM_001030 |
| | | (metallopanstimulin 1) | |
| 89 | RPS19 | ribosomal protein S19 | NM_001022 |
| 90 | RPS16 | ribosomal protein S16 | NM_001020 |
| 91 | MORF4L2 | mortality factor 4 like 2 | NM_012286 |
| 92 | RPL22 | ribosomal protein L22 | NM_000983 |
| 93 | RPS2 | ribosomal protein S2 | NM_002952 |
| 94 | RPLP2 | ribosomal protein, large, P2 | NM_001004 |
| 95 | RPL7A | ribosomal protein L7a | NM_000972 |
| 96 | RPL7 | ribosomal protein L7 | NM_000971 |
| 97 | RPS18 | ribosomal protein S18 | NM_022551 |
| 98 | HNRPH1 | Heterogeneous nuclear | NM_005520 |
| | | ribonucleoprotein H1 (H) | |
| 99 | ZNF160 | zinc finger protein 160 | NM_198893 |
| 100 | RPS25 | ribosomal protein S25 | NM_001028 |
| 101 | PGF | Placental growth factor, | NM_002632 |
| | | vascular endothelial growth | |
| | | factor-related protein | |
| 102 | SPG21 | spastic paraplegia 21 | NM_016630 |
| | | (autosomal recessive, Mast | |
| | | syndrome) | |
| 103 | RPL9 | ribosomal protein L9 | NM_000661 |
| 104 | PLEKHA5 | Pleckstrin homology domain | NM_019012 |
| | | containing, family A member 5 | |
| 105 | PRR11 | proline rich 11 | BC008669 |
| 106 | CTNNB1 | catenin (cadherin-associated | NM_001904 |
| | | protein), beta 1, 88 kDa | |
| 107 | NFKBIA | nuclear factor of kappa light | NM_020529 |
| | | polypeptide gene enhancer in | |
| | | B-cells inhibitor, alpha | |
| 108 | GTSE1 | G-2 and S-phase expressed 1 | NM_016426 |
| 109 | ATP8B1 | ATPase, Class I, type 8B, | NM_005603 |
| | | member 1 | |
| 110 | TMED2 | transmembrane emp24 domain | BC025957 |
| | | trafficking protein 2 | |
| 111 | RPS4X | ribosomal protein S4, X- | NM_001007 |
| | | linked | |
| 112 | MUC3B | mucin 3B, cell surface | XM_168578 |
| | | associated | |
| 113 | TTLL12 | tubulin tyrosine ligase-like | BC001070 |
| | | family, member 12 | |
| 114 | FTL | ferritin, light polypeptide | NM_000146 |
| 115 | TSPAN13 | Tetraspanin 13 | BC033863 |
| 116 | PTP4A2 | protein tyrosine phosphatase | NM_003479 |
| | | type IVA, member 2 | |
| 117 | EGLN3 | egl nine homolog 3 (C. elegans) | NM_022073 |
| 118 | ROCK2 | Rho-associated, coiled-coil | NM_004850 |
| | | containing protein kinase 2 | |
| 119 | NDRG1 | N-myc downstream regulated | NM_006096 |
| | | gene 1 | |
| 120 | GTPBP1 | GTP binding protein 1 | NM_004286 |
| 121 | RPL13 | ribosomal protein L13 | NM_000977 |
| 122 | CIDEC | cell death-inducing DFFA-like | BC016851 |
| | | effector c | |
| 123 | SIRT3 | sirtuin (silent mating type | NM_012239 |
| | | information regulation 2 | |
| | | homolog) 3 (S. cerevisiae) | |
| 124 | LAPTM4A | lysosomal-associated protein | NM_014713 |
| | | transmembrane 4 alpha | |
| 125 | NOS1 | nitric oxide synthase 1 | NM_000620 |
| | | (neuronal) | |
| 126 | COQ10B | coenzyme Q10 homolog B | NM_025147 |
| | | (S. cerevisiae) | |
| 127 | SAT | spermidine/spermine N1 | NM_002970 |
| | | acetyltransferase | |
| 128 | C1orf107 | chromosome 1 open reading | NM_014388 |
| | | frame 107 | |
| 129 | TXN | thioredoxin | NM_003329 |
| 130 | SLC7A1 | solute carrier family 7 | NM_003045 |
| | | (cationic amino acid | |
| | | transporter, y+ | |
| | | system), member 1 | |
| 131 | SLC7A7 | solute carrier family 1 | NM_006671 |
| | | (glutamate transporter), | |
| | | member 7 | |
| 132 | NTRK2 | neurotrophic tyrosine kinase, | NM_006180 |
| | | receptor, type 2 | |
| 133 | GSTA1 | Glutathione S-transferase A1 | NM_145740 |
| 134 | PTP4A3 | protein tyrosine phosphatase | NM_032611 |
| | | type IVA, member 3 | |

TABLE 38

| | Gene | | |
|---|---|---|---|
| | symbol | Gene Title | Acc No. |
| 51 | SEPP1 | selenoprotein P, plasma, 1 | NM_005410 |
| 52 | RPL27A | ribosomal protein L27A | NM_000990 |
| 53 | ATP1B1 | ATPase, Na+/K+ transporting, | NM_001677 |
| | | beta 1 polypeptide | |
| 54 | EEF1A1 | eukaryotic translation | NM_001402 |
| | | elongation factor 1 alpha 1 | |
| 55 | SFN | stratifin | NM_006142 |
| 56 | RPS11 | ribosomal protein S11 | NM_001015 |
| 57 | RPL23 | ribosomal protein L23 | NM_000978 |
| 59 | TPT1 | tumor protein, translationally- | NM_003295 |
| | | controlled 1 | |
| 60 | RPL41 | ribosomal protein L41 | NM_021104 |
| 61 | RPS29 | ribosomal protein S29 | NM_001032 |
| 62 | RPL38 | ribosomal protein L38 | NM_000999 |
| 63 | B2M | beta-2-microglobulin | NM_004048 |
| 64 | CFL1 | cofilin 1 (non-muscle) | NM_005507 |
| 65 | RPL31 | ribosomal protein L31 | NM_000993 |
| 67 | TMSB10 | thymosin, beta 10 | NM021103 |
| 68 | RPL39 | ribosomal protein L39 | NM_001000 |
| 69 | HMGB1 | high-mobility group box 1 | NM_002128 |
| 71 | RPS20 | ribosomal protein S20 | NM_001023 |
| 72 | ARF6 | ADP-ribosylation factor 6 | NM_001663 |
| 73 | RPS21 | ribosomal protein S21 | NM_001024 |
| 74 | ETF5A | Eukaryotic translation | NM_001970 |
| | | initiation factor 5A | |
| 75 | RPL30 | ribosomal protein L30 | NM_000989 |
| 76 | RPL23A | ribosomal protein L23a | NM_000984 |
| 78 | RPL27 | ribosomal protein L27 | NM_000988 |
| 79 | CEACAM5 | carcinoembryonic antigen- | NM_004363 |
| | | related cell adhesion molecule 5 | |
| 80 | RPS24 | ribosomal protein S24 | NM_001026 |
| 81 | MARCKS | Myristoylated alanine-rich | NM_002356 |
| | | protein kinase C substrate | |
| 86 | IL1RN | interleukin 1 receptor | NM_173842 |
| | | antagonist | |
| 88 | RPS27 | ribosomal protein S27 | NM_001030 |
| | | (metallopanstimulin 1) | |
| 89 | RPS19 | ribosomal protein S19 | NM_001022 |
| 90 | RPS16 | ribosomal protein S16 | NM_001020 |
| 91 | MORF4L2 | mortality factor 4 like 2 | NM_012286 |
| 92 | RPL22 | ribosomal protein L22 | NM_000983 |
| 93 | RPS2 | ribosomal protein S2 | NM_002952 |
| 94 | RPLP2 | ribosomal protein, large, P2 | NM_001004 |
| 97 | RPS18 | ribosomal protein S18 | NM_022551 |
| 98 | HNRPH1 | Heterogeneous nuclear | NM_005520 |
| | | ribonucleoprotein H1 (H) | |
| 100 | RPS25 | ribosomal protein S25 | NM_001028 |
| 103 | RPL9 | ribosomal protein L9 | NM_000661 |
| 106 | CTNNB1 | catenin (cadherin-associated | NM_001904 |
| | | protein), beta 1, 88 kDa | |
| 107 | NFKBIA | nuclear factor of kappa light | NM_020529 |
| | | polypeptide gene enhancer in B- | |
| | | cells inhibitor, alpha | |
| 110 | TMED2 | transmembrane emp24 domain | BC025957 |
| | | trafficking protein 2 | |
| 114 | FTL | ferritin, light polypeptide | NM_000146 |
| 115 | TSPAN13 | Tetraspanin 13 | BC033863 |
| 116 | PTP4A2 | protein tyrosine phosphatase | NM_003479 |
| | | type IVA, member 2 | |
| 117 | EGLN3 | egl nine homolog 3 (C. elegans) | NM_022073 |
| 119 | NDRG1 | N-myc downstream regulated gene 1 | NM_006096 |
| 120 | GTPBP1 | GTP binding protein 1 | NM_004286 |
| 121 | RPL13 | ribosomal protein L13 | NM_000977 |
| 122 | CIDEC | cell death-inducing DFFA-like | BC016851 |
| | | effector c | |
| 124 | LAPTM4A | lysosomal-associated protein | NM_014713 |
| | | transmembrane 4 alpha | |
| 125 | NOS1 | nitric oxide synthase 1 | NM_000620 |
| | | (neuronal) | |
| 126 | COQ10B | coenzyme Q10 homolog B | NM_025147 |
| | | (S. cerevisiae) | |
| 127 | SAT | spermidine/spermine N1- | NM_002970 |
| | | acetyltransferase | |
| 129 | TXN | thioredoxin | NM_003329 |

The genes selected as mentioned above highly include ribosomal protein genes (Table 39).

TABLE 39

| | Gene | | |
|---|---|---|---|
| | symbol | Gene Title | Acc No. |
| 52 | RPL27A | ribosomal protein L27A | NM_000990 |
| 56 | RPS11 | ribosomal protein S11 | NM_001015 |
| 57 | RPL23 | ribosomal protein L23 | NM_000978 |
| 60 | RPL41 | ribosomal protein L41 | NM_021104 |
| 61 | RPS29 | ribosomal protein S29 | NM_001032 |
| 62 | RPL38 | ribosomal protein L38 | NM_000999 |
| 65 | RPL31 | ribosomal protein L31 | NM_000993 |
| 66 | RPS3A | ribosomal protein S3A | NM_001006 |
| 68 | RPL39 | ribosomal protein L39 | NM_001000 |
| 71 | RPS20 | ribosomal protein S20 | NM_001023 |
| 73 | RPS21 | ribosomal protein S21 | NM_001024 |
| 75 | RPL30 | ribosomal protein L30 | NM_000989 |
| 76 | RPL23A | ribosomal protein L23a | NM_000984 |
| 78 | RPL27 | ribosomal protein L27 | NM_000988 |
| 80 | RPS24 | ribosomal protein S24 | NM_001026 |
| 84 | RPS10 | ribosomal protein S10 | NM_001014 |
| 88 | RPS27 | ribosomal protein S27 | NM_001030 |
| | | (metallopanstimulin 1) | |
| 89 | RPS19 | ribosomal protein S19 | NM_001022 |
| 90 | RPS16 | ribosomal protein S16 | NM_001020 |
| 92 | RPL22 | ribosomal protein L22 | NM_000983 |
| 93 | RPS2 | ribosomal protein S2 | NM_002952 |
| 94 | RPLP2 | ribosomal protein, large, P2 | NM_001004 |
| 95 | RPL7A | ribosomal protein L7a | NM_000972 |
| 96 | RPL7 | ribosomal protein L7 | NM_000971 |
| 97 | RPS18 | ribosomal protein S18 | NM_022551 |
| 100 | RPS25 | ribosomal protein S25 | NM_001028 |
| 103 | RPL9 | ribosomal protein L9 | NM_000661 |
| 111 | RPS4X | ribosomal protein S4, X-linked | NM_001007 |
| 121 | RPL13 | ribosomal protein L13 | NM_000977 |

TABLE 30

| | | | | |
|---|---|---|---|---|
| No. | Gene name | GenBank ID | probe (5′→3′) | Sequence ID No. |

| | | | | |
|---|---|---|---|---|
| 51 | SEPP1 | NM_005410 | CCATAGTCAATGATGGTTTAATAGGTAAACCAAACCCTATAAACCTGACCTCCTTTATGG | SEQ ID NO: 151 |
| 52 | RPL27A | NM_000990 | CCAACTGTCAACCTTGACAAATTGTGGACTTTGGTCAGTGAACAGACACGGGTGAATGCT | SEQ ID NO: 152 |
| 53 | ATP1B1 | NM_001677 | GAGTGTAAGGCGTACGGTGAGAACATTGGGTACAGTGAGAAAGACCGTTTTCAGGGACGT | SEQ ID NO: 153 |
| 54 | EEF1A1 | NM_001402 | CCACCCCACTCTTAATCAGTGGTGGAAGAACGGTCTCAGAACTGTTTGTTTCAATTGGCC | SEQ ID NO: 154 |
| 55 | SFN | NM_006142 | CTCTGATCGTAGGAATTGAGGAGTGTCCCGCCTTGTGGCTGAGAACTGGACAGTGG | SEQ ID NO: 155 |
| 56 | RPS11 | NM_001015 | TCATCCGCCGAGACTATCTGCACTACATCCGCAAGTACAACCGCTTCGAGAAGCG | SEQ ID NO: 156 |
| 57 | RPL23 | NM_000978 | ACATCCAGCAGTGGTCATTCGACAACGAAAGTCATACCGTAGAAAAGATGGCGTGTTTCT | SEQ ID NO: 157 |

TABLE 31

| | | | | | |
|---|---|---|---|---|---|
| No. | Gene name | Forward-Primer(5′→3′) | Sequence ID No. | Reverse-Primer(5′→3′) | Sequence ID No. |

| | | | | | |
|---|---|---|---|---|---|
| 51 | SEPP1 | AATTAGCAGTTTAGAATGGAGG | SEQ ID NO: 158 | CTGTATCCAATTCTGTACTGC | SEQ ID NO: 165 |
| 52 | RPL27A | TGGGCTGCCAACATGCCATC | SEQ ID NO: 159 | TGTAGTAGCCCGATCGCACC | SEQ ID NO: 166 |
| 53 | ATP1B1 | GGCAAGCGAGATGAAGATAAGG | SEQ ID NO: 160 | AGGTCCCATA CGTATGACAG | SEQ ID NO: 167 |
| 54 | EEF1A1 | AGACTATCCACCTTTGGGTCG | SEQ ID NO: 161 | GATGCATTGTTATCATTAACCAGTC | SEQ ID NO: 168 |
| 55 | SFN | TTGAGCGCACCTAACCACTGGT | SEQ ID NO: 162 | GAGAGGAAACATGGTCACACCCA | SEQ ID NO: 169 |
| 56 | RPS11 | ACATTCAGACTGAGCGTGCCTA | SEQ ID NO: 163 | GATCTGGACGTCCCTGAAGCA | SEQ ID NO: 170 |
| 57 | RPL23 | TTCAAGATGTCGAAGCGAGGAC | SEQ ID NO: 164 | TGTAATGGCAGAACCTTTCATCTCG | SEQ ID NO: 171 |

### Example 9

### RT-PCR and High Sensitivity Chip Analyses Using Cells Isolated from Stool Samples of Colon Cancer Patients

(1) RT-PCR Analyses

RT-PCR analyses were carried out for the 7 genes chosen in Example 8 using cell RNA isolated from 7 healthy subjects and 25 colon cancer patients. The 7 genes described above, their probes and primers are summarized in Table 30 and 31. The experimental procedures are the same as in Example 6 (i)-(v).

The results of the analysis for the 7 genes indicated that in the stool samples from 7 healthy subjects only 1 case was positive for 1 gene (No. 102). On the other hand, in the stool samples from 25 colon cancer patients, as a whole 64% ( 16/25) was positive for at least 1 gene. For the 9 cases in which β-actin mRNA was detected, indicating there were many cells, at least one gene was positive in about 90% ( 8/9) (Table 32).

TABLE 32

| | | No. 51 | No. 52 | No. 53 | No. 54 | No. 55 | No. 56 | No. 57 | Positive | |
|---|---|---|---|---|---|---|---|---|---|---|
| | | SEPP1 | RPL27A | ATP1B1 | EEF1A1 | SFN | RPS11 | RPL23 | judgment | β-actin |
| Healthy subject | 1 | | | | | | | | | ◯ |
| | 2 | | | | | | | | | ◯ |
| | 3 | | | | | | | | | |
| | 4 | | | | | | | | | ◯ |
| | 5 | | | | | | | ◯ | ◯ | ◯ |
| | 6 | | | | | | | | | |
| | 7 | | | | | | | | | |
| Colon cancer patient | 7 | ◯ | ◯ | ◯ | ◯ | ◯ | ◯ | ◯ | ◯ | ◯ |
| | 22 | | | | ◯ | | | ◯ | ◯ | |
| | 30 | | | | ◯ | | | | ◯ | ◯ |
| | 10 | | | | | | ◯ | | ◯ | |
| | 11 | | | | | | | | | |
| | 12 | | | | | | | | | ◯ |
| | 13 | ◯ | ◯ | | | | | | ◯ | |
| | 14 | | | | | | | | | |
| | 15 | ◯ | ◯ | ◯ | ◯ | ◯ | ◯ | ◯ | ◯ | ◯ |
| | 16 | | ◯ | | ◯ | | ◯ | ◯ | ◯ | |
| | 17 | ◯ | ◯ | ◯ | ◯ | ◯ | ◯ | ◯ | ◯ | ◯ |
| | 18 | | ◯ | | ◯ | | ◯ | ◯ | ◯ | ◯ |
| | 2 | | | | | | | | | |
| | 3 | | | | ◯ | | ◯ | ◯ | ◯ | ◯ |
| | 4 | | | | | | | | | |
| | 6 | | ◯ | | | | | | ◯ | |
| | 20 | | | | | ◯ | ◯ | | ◯ | |
| | 23 | | | | | | | | | |
| | 24 | | ◯ | | ◯ | | | | ◯ | ◯ |
| | 25 | ◯ | ◯ | | ◯ | ◯ | ◯ | ◯ | ◯ | ◯ |
| | 26 | | | | | | | ◯ | ◯ | |
| | 27 | | | | | | | | | |
| | 28 | | | | | | | | | |
| | 31 | | ◯ | | ◯ | | | | ◯ | |
| | 32 | | | | | | | | | |

(2) Chip Analysis

Probe DNAs (SEQ ID NO: 151-157) for detecting the 7 genes chosen in Example 8 were synthesized and DNA microarrays were prepared as described in Example 4 I. Using this DNA microarray, expression analysis was carried out in a similar manner to Example 7 after performing multiplex PCR amplification by incorporated label, the targets of which are the 7 genes, using primers shown in Table 31. The multiplex PCR was performed by adding all the 7 kinds of primers to a PCR tube. The substrates were mixed with Cy3-dUTP to standardize PCR products. The PCR reaction mixture composition, temperature cycle protocol and method for hybridizing to the DNA microarray are similar to those in Example 7. Fluorescent luminescence values of each probe to the 32 labeled samples are shown in Table 33.

TABLE 33

| | | No. 51 | No. 52 | No. 53 | No. 54 | No. 55 | No. 56 | No. 57 |
|---|---|---|---|---|---|---|---|---|
| | | SEPP1 | RPL27A | ATP1B1 | EEF1A1 | SFN | RPS11 | RPL23 |
| Healthy subject | 1 | 2.5 | 2.1 | 2.5 | 3.0 | 2.7 | 4.0 | 1.8 |
| | 2 | 3.1 | 6.0 | 5.5 | 29.4 | 3.7 | 7.1 | 20.6 |
| | 3 | 2.3 | 2.0 | 1.5 | 3.1 | 2.6 | 4.1 | 1.7 |
| | 4 | 2.3 | 2.1 | 2.7 | 17.8 | 2.6 | 3.0 | 2.3 |
| | 5 | 2.2 | 4.6 | 1.4 | 1.4 | 0.9 | 1.4 | 28.3 |
| | 6 | 1.7 | 1.7 | 2.3 | 2.0 | 1.9 | 2.8 | 1.9 |
| | 7 | 2.3 | 5.4 | 3.1 | 6.1 | 3.0 | 12.0 | 9.3 |
| Colon cancer patient | 7 | 478.8 | 1090.3 | 341.5 | 1393.3 | 34.1 | 630.9 | 906.9 |
| | 22 | 2.4 | 2.2 | 2.2 | 159.6 | 2.5 | 2.8 | 42.4 |
| | 30 | 2.3 | 2.8 | 3.0 | 60.6 | 2.2 | 4.7 | 26.7 |
| | 10 | 2.2 | 2.1 | 2.2 | 21.0 | 2.0 | 80.0 | 0.9 |
| | 11 | 0.7 | 0.1 | 0.1 | 0.1 | 0.1 | 2.9 | 0.1 |
| | 12 | 1.9 | 1.0 | 1.3 | 2.8 | 5.6 | 6.9 | 0.8 |
| | 13 | 97.0 | 221.6 | 6.3 | 9.7 | 3.4 | 5.0 | 1.4 |
| | 14 | 0.1 | 0.1 | 0.1 | 0.1 | 0.1 | 2.3 | 0.1 |
| | 15 | 681.8 | 823.1 | 270.4 | 706.3 | 67.0 | 458.1 | 441.4 |
| | 16 | 1.0 | 437.2 | 1.4 | 204.8 | 1.6 | 206.1 | 236.8 |
| | 17 | 826.2 | 918.3 | 220.4 | 1585.5 | 39.4 | 726.0 | 629.3 |
| | 18 | 1.9 | 638.1 | 18.7 | 1181.6 | 0.8 | 562.8 | 563.8 |
| | 2 | 1.4 | 6.0 | 4.4 | 7.8 | 2.1 | 4.2 | 0.8 |
| | 3 | 18.6 | 1.5 | 1.5 | 157.7 | 3.7 | 65.9 | 65.7 |
| | 4 | 0.4 | 0.8 | 0.8 | 6.2 | 1.2 | 3.0 | 6.3 |
| | 6 | 2.0 | 30.1 | 1.9 | 2.9 | 1.9 | 4.2 | 0.6 |
| | 20 | 2.5 | 35.8 | 1.8 | 2.3 | 34.0 | 42.6 | 1.1 |
| | 23 | 1.5 | 1.4 | 1.6 | 1.4 | 1.1 | 6.3 | 1.1 |
| | 24 | 4.5 | 144.8 | 1.9 | 81.1 | 4.8 | 10.5 | 44.1 |
| | 25 | 239.9 | 51.9 | 1.8 | 273.3 | 8.3 | 165.0 | 166.7 |
| | 26 | 2.3 | 1.6 | 2.1 | 2.0 | 1.4 | 1.6 | 55.8 |
| | 27 | 2.9 | 2.1 | 1.9 | 3.1 | 1.9 | 5.7 | 1.0 |
| | 28 | 2.1 | 1.0 | 2.2 | 1.3 | 2.9 | 5.1 | 0.7 |
| | 31 | 2.3 | 136.9 | 1.6 | 92.5 | 1.4 | 25.5 | 0.5 |
| | 32 | 1.3 | 1.4 | 0.8 | 0.5 | 1.2 | 1.7 | 0.4 |

(3) Experimental Results

The presence/absence of the expression of the 7 genes in each case is summarized in Table 34. In fluoroluminance values in Table 33, a value of 30 or above was defined as positive. If one gene or more among the 7 genes was positive, the judgment was “o”. The presence/absence of the expression of β-actin and cytodiagnosis are the same as Table 22 in Example 7.

The results for the 7 genes indicated that all of the 7 stool samples from healthy subjects were negative. On the other hand, in 25 stool samples from colon cancer patients, the positivity rate was 64% ( 16/25) as a whole. For the cases in which actin mRNA was detected, indicating there were many cells, at least one gene was positive in about 90% ( 8/9). In the cytodiagnosis 6/25 were positive, and in comparison with this result, the result of the expression analysis using the 7 genes set of the present invention would be significant data. Also the positive predictive rate is 100% and is superior to that of the occult blood test for stool.

TABLE 34

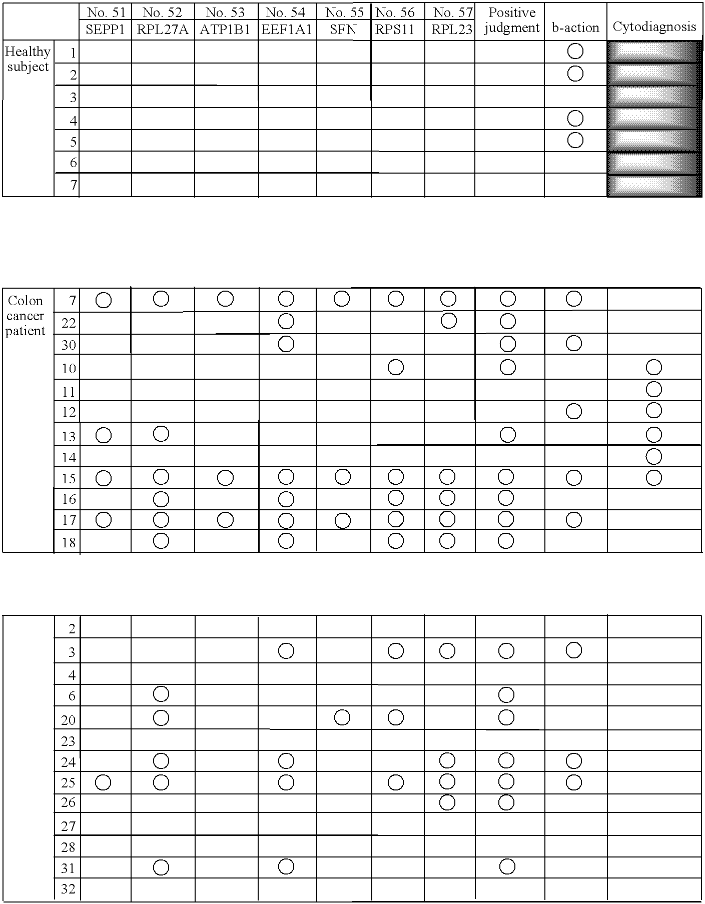

The results of Table 32 and Table 34 indicated that the results of RT-PCR of (1) and DNA microarray analysis of (2) were almost the same. Thus, it has been shown that diagnosis of colon cancer is possible by preparing samples by multiplex PCR and analyzing by microarray using small number of cells recovered from stool.

Further, the results combined with the results shown in Example 7 are shown in Table 35. Here, the targeting genes were 11 genes because the No. 38 gene with relatively low detection rate was eliminated. The results of the analysis for the 11 genes indicated that in the stool samples from 7 healthy subjects only 1 case was positive for 1 gene. On the other hand, in the stool samples from 25 colon cancer patients, 20 cases were positive for at least 1 gene, and the positivity rate was 80% ( 20/25). For the cases in which β-actin mRNA was detected, indicating there were many cells, were positive in 100% ( 9/9).

Table 36 shows comparison of the results of cytodiagnosis with that of the expression analysis by microarray. The 6 cytodiagnosis positive cases were all detected by microarray, too. Further, for the 19 cytodiagnosis negative cases, 14 cases, which was about 70%, could be diagnosed as positive, indicating that the results by microarray was far superior to that by cytodiagnosis. As described above, it has been confirmed that the gene set of the present invention is effective for diagnosis of colon cancer.

Further, the same samples were examined for positivity for the genes listed in Table 35 except for No. 50. As a result, the 7 samples of healthy subjects were negative for all the genes. On the other hand, 19 samples among the 25 samples of colon cancer patients were positive for at least one gene, hence the positivity rate being 76% ( 19/25).

Please note in this regard that among the 25 samples of colon cancer subjects, 8 samples of Stage I patients showed a positivity rate of 63% (⅝), 6 samples of Stage II patients showed a positivity rate of 83% (⅚), and 9 samples of Stage IIIa patients showed a positivity rate of 89% ( 8/9). Thus, it has been confirmed that the gene set of the present invention can detect not only advanced cancer but also early cancer.

TABLE 35

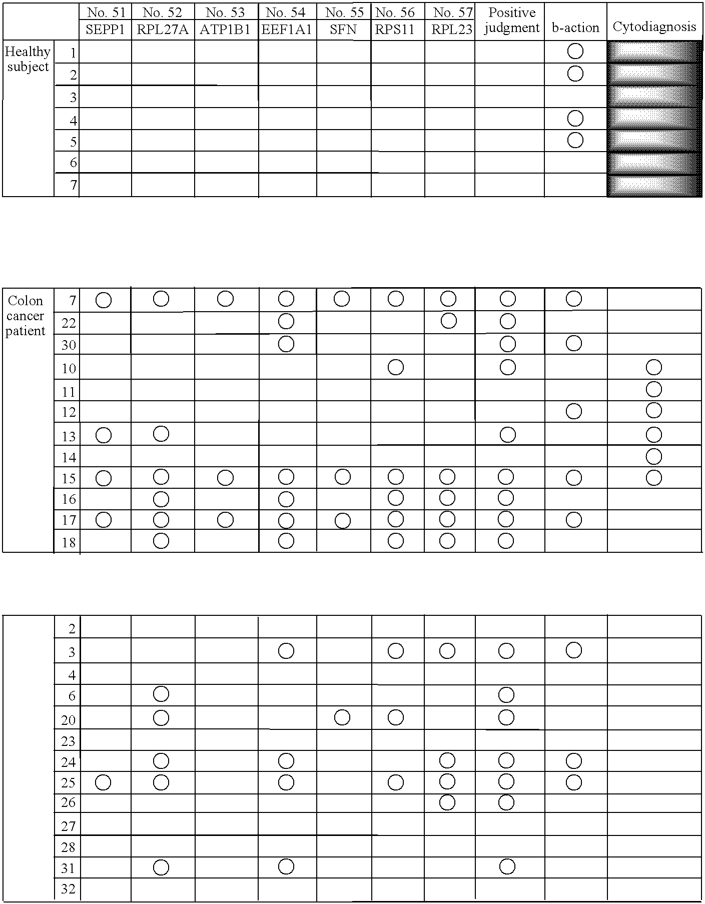

TABLE 36
Comparison with cytodiagnosis results

| | | | |
|---|---|---|---|
| Cancer patient | − (negative) | 14/19 (74%) | 20/25 (80%) |
| | + (positive) | 6/6 (100%) | |
| Healthy subject | | | 1/7 (14%) |

### Example 10

### RT-PCR Analysis Using Cells Separated from Stool Samples of Colon Cancer Subjects

Of the 84 genes chosen in Example 9, 4 genes were randomly chosen, and RT-PCR analysis was effected for each of the 4 genes using RNA in cells separated from stool samples of 4 healthy subjects and 4 colon cancer subjects. The primers for the above 4 genes are listed in Table 40.

TABLE 40

| | | | |
|---|---|---|---|
| No. | gene | Forward-Primer (5′→3′) | Reverse-Primer (5′→3′) |

| | | | |
|---|---|---|---|
| 61 | RPS29 | AAAATTCGGCCAGGGTTCTC | GAGCATTTAGTCCAACTTAATGAAA |
| 62 | RPL38 | TCGCCATGCCTCGGAAAATTGA | GGACTGCTTCAGTTTCTCTG |
| 71 | RPS20 | GCCTACCAAGACTTTGAGAATC | GACTTAAGCATCTGCAATGGTG |
| 129 | TXN | GATGACTGTCAGGATGTTGCT | CTTTTCCTTATTGGCTCCAGAA |

The same steps as of (i) to (v) in Example 6 were conducted, followed by step (vi) described below.

(vi) PCR Amplification Reaction

Using as template the recovered first strand cDNA solution, PCR amplification was effected for the selected 4 genes. As primers, the primer set listed in Table 40 was used.

For the PCR reaction, a reaction solution shown in table 41 was prepared by using a PCR kit AccuPrime Taq supplied by Invitrogen Corporation. PCR amplification reaction was carried out for the prepared reaction mixture by using a commercially available thermal cycler according to the temperature cycle protocol shown in Table 42. The reaction mixture after completion of the PCR was stored at 4° C.

TABLE 41
Reaction mixture composition

| Component | Composition | |
|---|---|---|
| Invitrogen, AccuPrimer Taq | 1.0 | μl |
| 10x AccuPrime PCR Buffer | 2.5 | μl |
| Template DNA (cDNA solution) | 1.0 | μ1 |
| Forward Primer (F) | 25 | pmol |
| Reverse Primer (R) | 25 | pmol |
| Distilled water | Optional | |
| Total | 25 | μl |

TABLE 42
Temperature condition for PCR amplification

| Step | Temperature | | Holding time | Repeat No. |
|---|---|---|---|---|
| 1 | 95° C. | | 5 min. | |
| 2 | 95° C. | (denaturation) | 30 sec. | 30 cycles |
| 3 | 58° C. | (annealing) | 30 sec. | |
| 4 | 72° C. | (extension) | 40 sec. | |
| 5 | 72° C. | | 10 min. | |

(3) Experimental Results

Using 10 μl from each of the resulting PCR products, 1.5% agarose gel electrophoresis was conducted, and this was stained with EtBr solution. As a result, stool samples of four healthy subjects were all negative for each of the 4 genes while stool samples of four colon cancer patients were positive for at least one gene (Table 43). Accordingly, it was found that these genes were suitable for screening of colon cancer.

TABLE 43

| | | colon cancer | | | | | | | |
|---|---|---|---|---|---|---|---|---|---|
| | | subject | | | | healthy subject | | | |
| | gene | T1 | T2 | T3 | T4 | N1 | N2 | N3 | N4 |
| 61 | RPS29 | + | + | + | − | − | − | − | − |
| 62 | RPL38 | + | + | + | − | − | − | − | − |
| 71 | RPS20 | + | + | + | + | − | − | − | − |
| 129 | TXN | + | − | + | + | − | − | − | − |

| | | | | | | | | |
|---|---|---|---|---|---|---|---|---|
| judgment | + | + | + | + | − | − | − | − |

As described above, the 57 genes of the present invention are useful as diagnostic markers for colon cancer. Thus, the probes, primers and samples fixed to solid phase of the present invention can be utilized for early diagnosis of colon cancer.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2005-360974, filed Dec. 14, 2005 which is hereby incorporated by reference herein in its entirety.

What is claimed is:

1. A method for screening colon cancer cells in a sample by analyzing an amount of expression of at least 2 or more genes, or products thereof, selected from the group of genes listed in Table 1 and Table 30.

2. A method for screening colon cancer cells in a sample by analyzing an amount of expression of at least 2 or more genes, or products thereof, selected from the group of genes listed in Table 1.

3. A method for screening colon cancer cells in a sample by analyzing an amount of expression of at least 2 or more genes, or products thereof, selected from the group of genes listed in Table 26.

4. A method for screening colon cancer cells in a sample by analyzing an amount of expression of at least 2 or more genes, or products thereof, selected from the group of genes listed in Table 28.

5. The method according to claim 1, wherein said sample is a smear of stool.

6. A method for screening colon cancer cells in a stool sample by analyzing an amount of expression of at least 2 or more genes, or products thereof, selected from the group of genes listed in Table 30.

7. The method according to claim 1, wherein an amount of expression of a gene is analyzed by using an amount of a mRNA in a sample.

8. The method according to claim 1, wherein an expression amount of a gene product is analyzed by using an antibody against the gene product.

9. A method for examination of colon cancer using the method according to claim 1.

10. The method for examination according to claim 9, wherein the colon cancer is early colon cancer.

11. A primer for amplifying specifically any one of the genes listed in Table 1 and Table 30, said primer comprising an oligonucleotide having any one of the base sequences of SEQ ID NOs: 51-150 and 158-171, which may contain deletion, substitution or addition of one or a few bases.

12. A probe for detecting any one of the genes listed in Table 1 and Table 30 by hybridizing specifically with the genes, said probe comprising an oligonucleotide having any one of the base sequences of SEQ ID NOs: 1-50 and 151-157, which may contain deletion, substitution or addition of one or a few bases.

13. A sample fixed on a solid phase, wherein the probe according to claim 12 is fixed on a solid carrier.

14. A gene detection kit for at least 2 or more genes selected from the group of genes listed in Table 1 and Table 30, comprising the primer according to claim 11, the probe according to claim 12 and/or the sample fixed on a solid phase according to claim 13.

15. A gene marker set for testing colon cancer comprising at least 2 or more genes selected from the group of genes listed in Table 1 and Table 30.

16. A method for screening cancer cells in stool, comprising steps of:
(i) selecting a group of genes satisfying the requirements (1) to (3) given below, based on a result of expression analysis in cancer cells and live normal cells:
(1) expression is observed in live normal cells;
(2) expression is observed in live cancer cells; and
(3) expression is not observed in dead cells; and
(ii) analyzing expression of the selected genes in stool to thereby screening cancer cells without separating normal cells from the cancer cells.

17. The method for screening cancer cells according to claim 16, wherein the cancer cells are colon cancer cells.

18. The method for screening cancer cells according to claim 16, wherein a gene expressed in peripheral blood is excluded in the selection step.

19. The method for screening cancer cells according to claim 16, wherein the selected genes include at least two selected from the 84 genes listed in Table 37.

20. The method for screening cancer cells according to claim 16, wherein the selected genes include at least two selected from the 48 genes listed in Table 38.

21. The method for screening cancer cells according to claim 16, wherein the gene selection is performed by selecting a libosomal protein gene as candidate and comparing expression of the selected genes in cells collected from a stool sample of a healthy subject and in cells collected from a stool sample of a cancer patient.

22. A method for screening cancer cells in stool, comprising detecting expression of a part or all of a group of libosomal protein genes in cells in stool.

23. The method for screening cancer cells in stool according to claim 22, wherein a part of the group of libosomal genes are the genes listed in Table 39.
